(19) United States
(12) Patent Application Publication
(10) Pub. No.: US 2020/0345867 A1
(43) Pub. Date: Nov. 5, 2020

(54) ORGAN DIRECTED GENE DELIVERY

(71) Applicant: The Johns Hopkins University, Baltimore, MD (US)

(72) Inventors: Florin M. Selaru, Catonsville, MD (US); Vivek Kumbhari, Baltimore, MD (US); Ling Li, Baltimore, MD (US)

(21) Appl. No.: 16/864,130

(22) Filed: Apr. 30, 2020

Related U.S. Application Data
(60) Provisional application No. 62/840,474, filed on Apr. 30, 2019.

(51) Int. Cl. A61K 48/00; C12N 15/63; A61P 1/18; A61P 1/16

(52) U.S. Cl. CPC A61K 48/0083; C12N 15/63; A61K 9/0019; A61P 1/16; A61K 48/0075; A61P 1/18

(57) ABSTRACT

Intrabiliary hydrodynamic injection of nucleic acid for in vivo gene therapy for treatment of liver or pancreas disease and other disorders.

This application claims benefit of priority to U.S. Provisional Application No. 62/840,474 filed on Apr. 30, 2019, the entire contents of which application is incorporated herein by reference.

### STATEMENT OF GOVERNMENTAL INTEREST

This invention was made with government support under grant nos. DK090154, R01CA190040, R01EB017742 awarded by the National Institutes of Health. The government has certain rights in the invention.

### BACKGROUND OF THE INVENTION

The liver is affected in many acquired and inherited gene disorders. Devastating single gene disorders such as alpha-1 antitrypsin deficiency, cystic fibrosis, and many others could theoretically be treated by inserting a corrected copy of the defective gene into affected liver cells. This presents an opportunity for the application of liver-targeted gene therapy, where the replacement of single gene has been shown to have a significant clinical impact.¹ However, a technically simple, free of fatal complications, liver-specific method to deliver gene therapy does not currently exist. The lack of such a method is a major drawback to the effective treatment of millions of patients, many of whom are children.² Previous attempts at treatment of some of these disorders highlighted the potentially catastrophic side effects associated with the delivery vehicle, as well as with the method of delivery.^{3,4} There is need for the development of a clinical-grade, simple, safe, and efficient in vivo nucleic acid delivery system.

### SUMMARY

In one aspect, we now provide intra-biliary delivery of nucleic acid via hydrodynamic administration.

In one embodiment, methods are provided to treat or prevent kidney liver or pancreas disease by genetic therapy. In particular embodiments, methods are provided to treat or prevent hemophilia and other diseases and disorders as disclosed herein by genetic therapy. The method includes the steps of delivering a vector comprising a nucleic acid sequence that ameliorates a kidney liver or pancreas disease or other disease or disorder such as hemophilia to a subject having or prone of getting a liver or pancreas disease or other disease or disorder such as hemophilia.

Thus, in one aspect, methods are provided for transfecting cells of a subject in vivo, comprising administering at elevated pressure an effective amount of a nucleic acid expression cassette to the subject's biliary tree, liver or pancreas.

Preferably, the elevated pressure is measured during administering of the nucleic acid expression cassette. Also preferred is to modify the elevated pressure as desired during and/or subsequent to the administering based on real-time measuring of the elevated pressure.

Preferably, a nucleic acid expression cassette is delivered into a substantially closed system. For example, a vessel or organ of a subject, optionally together with a medical device or tool can be utilized to provide a substantially closed system where an elevated pressure of administered composition comprising the nucleic acid expression cassette.

In a preferred aspect, a composition comprise a nucleic acid expression cassette is administered within a subject's biliary tree which can provide a substantially closed system that can readily enable elevating pressure of the administered composition. In one aspect, a nucleic acid expression cassette is administered into or within a subject's common bile duct. In one aspect, a nucleic acid expression cassette is administered into or within a subject's hepatic duct. In another aspect, a nucleic acid expression cassette is administered into or within a subject's pancreatic duct.

In a further aspect, a nucleic acid expression cassette can be administrations under conditions based on one or more of 1) measured volume of a subject's biliary tree or volume/characteristics of other administration site; and 2) measured target organ characteristics.

In a further aspect, methods for transfecting cells of a subject in vivo are provided that include at elevated pressure an effective amount of a nucleic acid expression cassette to the subject, wherein the elevated pressure is measured and assessed in real-time during and/or subsequent to administering of the nucleic acid expression cassette. Preferably, the elevated pressure is modified as desired during and/or subsequent to the administering based on real-time measuring of the elevated pressure. Again, in preferred aspects, the nucleic acid expression cassette is delivered into a substantially closed system, such as a subject's biliary tree, liver, or pancreas.

In a further aspect, methods of treating or preventing liver or pancreas disease by genetic therapy are provided and comprises: delivering a vector comprising a nucleic acid sequence that ameliorates a liver or pancreas disease to a subject having or prone of getting a liver or pancreas disease; creating a substantially closed space; injecting the vector under required pressure and quantity into the closed space so that the vector is transferred to the cytoplasm of cells of the liver or pancreas; and expressing the nucleic acid sequence and treating or preventing a liver or pancreas disease in the subject.

As referred to herein, the term substantially closed space refers to an in vivo space where an elevated pressure can be induced upon delivery of a composition (e.g. fluid composition) that contain a nucleic acid expression cassette. In the present methods, the pressure will be elevated above the initial introduction or infusion pressure of the fluid composition, e.g. at least about a 5, 10, 15, 20, 30, 40, 50, 60, 70, 80, 90, 100, 120, 140, 160, 180 or 200 percent increase, or even higher such as 5, 8, 10, 15, 20, 25, 30, 40, 50 or 60 times increase, in pressure relative to the pressure that the fluid composition is initially introduced to the space such as by injection. It is possible that administered fluid (e.g. fluid composition comprising a nucleic acid expression cassette) may leak from the closed space during an administration protocol, such as during injection of the fluid composition to the target site, or following such injection.

In certain preferred aspects of the present methods, a device that can facilitate creating or maintaining a substantially closed space may be utilized with the administration of a nucleic acid expression cassette. For a device can be utilized that include a balloon that can establish or facilitate maintaining a substantially closed space during administration of a nucleic acid expression cassette.

In particular aspects, an endoscopic retrograde cholangiopancreatography (ERCP) that comprises balloon may be utilized with the administration of a nucleic acid expression cassette. The ERCP also may contain the nucleic acid expression cassette for administration. In use, endoscope of the ERCP may be positioned through the mouth, the stomach, duodenum, and the duct of the pancreas or the liver of a subject; and the balloon of the ERCP may be opened to thereby create an in vivo substantially closed space which can facilitate establishing and maintaining an elevated pressure of the nucleic acid expression cassette during the course of an administration protocol.

Suitable nucleic acid sequences used in the present invention may encodes a protein or a peptide or a second nucleic acid selected from the group consisting of a shRNA, mRNA, and a combination thereof. The protein or peptide may be an antibody. In some embodiments, the nucleic acid sequence encodes a protein that treats cirrhosis, hemophilia, cystic fibrosis, urea cycle enzyme alterations, or a functional part thereof. In some embodiments, the nucleic acid sequence encodes a protein that treats a pancreas disease or a functional part thereof. Suitable vectors include a virus, a plasmid, or both, as examples. An example of a virus used in the present invention is AAV. In some embodiments, a plasmid may comprise an SB transposon comprising the nucleic acid sequence described above. In some embodiments, the methods of the present invention may include an additional step of removing the remaining vector through the endoscope.

In some embodiments, a vector comprising any of the expression cassettes disclosed herein is provided.

The present methods and systems are particularly useful to treat or prevent liver-related diseases and disorders including for example, hemophilia, alpha-1 antitrypsin deficiency, familial hypercholesterolemia, Wilson's disease, Crigler-Najjar Syndrome, methymalonic academia, and/or ornithine transcarbamylase deficiency.

In particular aspect, the present methods and systems are used to treat hemophilia, including hemophilia B, for example nucleic acid expressing Factor IX is administered

The present methods and systems are particularly useful to treat or prevent pancreas-related diseases and disorders including for example, Type I DM, single gene disorders causing pancreatitis: CFTR, CLDN2, CPA1, PRSS1 and SPINK1.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as Indoleamine 2,3-dioxygenase (an oxidoreductase) catalyzing the degradation of the essential amino acid tryptophan (trp) to N-formyl-kynurenine.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a VH domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “disease” is meant any condition or disorder that damages or interferes with the normal function of a cell, tissue, or organ. Examples of diseases include cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “ERCP” is meant Endoscopic retrograde cholangiopancreatography (ERCP) is a technique that combines the use of endoscopy and fluoroscopy to diagnose and treat certain problems of the biliary or pancreatic ductal systems. Through the endoscope, the physician can see the inside of the stomach and duodenum, and inject a contrast medium into the ducts in the biliary tree and pancreas so they can be seen on radiographs. ERCP is used primarily to diagnose and treat conditions of the bile ducts and main pancreatic duct, including gallstones, inflammatory strictures (scars), leaks (from trauma and surgery), and cancer. ERCP can be performed for diagnostic and therapeutic reasons, although the development of safer and relatively non-invasive investigations such as magnetic resonance cholangiopancreatography (MRCP) and endoscopic ultrasound has meant that ERCP is now rarely performed without therapeutic intent.

The term “express” refers to the ability of a gene to express the gene product including for example its corresponding mRNA or protein sequence (s).

As used herein, the term “expression cassette” or “nucleic acid expression cassette” refers to a DNA sequence that encodes and is capable of producing one or more desired expression products (RNA or protein). Production of such a desired expression product may require the presence of various expression control sequences operatively linked to the DNA sequence encoding that product. Such control sequences include a promoter, as well as other non-coding nucleotide sequences. An expression cassette may include none, some or all of these expression control sequences. If some or all of these expression control sequences are absent from the expression cassette, they are supplied by a vector into which the expression cassette is inserted.

As used herein, a “subject” means a human. The terms, “patient”, “individual” and “subject” are used interchangeably herein. A subject can be one who has been previously diagnosed with or identified as suffering from or having a condition in need of treatment (e.g., brain tumors) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition, e.g. a neurodegenerative condition, can be a subject suspected of having that condition, diagnosed as having that condition, already treated or being treated for that condition, not treated for that condition, or at risk of developing that condition.

The term “biliary tree” includes the common bile duct and hepatic duct. The pancreatic duct also may be included with the common bile duct and/or hepatic duct in certain aspects.

The term “polynucleotide” as used herein means a sequence of 20 or more nucleotides. A polynucleotide may RNA, DNA or a hybrid RNA or DNA molecule; and may be single stranded or double stranded. In certain embodiments, a polynucleotide is a single or double-stranded DNA molecule.

The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

“Immunoassay” is an assay that uses an antibody to specifically bind an antigen (e.g., a marker). The immunoassay is characterized by the use of specific binding properties of a particular antibody to isolate, target, and/or quantify the antigen.

The term, “obtaining” as in “obtaining an agent” includes synthesizing, purchasing, or otherwise acquiring the agent.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” is meant a negative alteration of at least 10%, 25%, 50%, 75%, or 100%.

A “reference” refers to a standard or control conditions such as a sample (human cells) or a subject that is a free, or substantially free, of an agent such as plasmid or transposon of the present invention.

A “reference sequence” is a defined sequence used as a basis for sequence comparison.

A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “specifically binds” is meant a compound or antibody that recognizes and binds a polypeptide of the invention, but which does not substantially recognize and bind other molecules in a sample, for example, a biological sample, which naturally includes a polypeptide of the invention.

As used herein, the term “subject” is intended to refer to any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and ^{e-100} indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

A “transgene” is used herein to conveniently refer to a polynucleotide or a nucleic acid that is intended or has been introduced into a cell or organism. Transgenes include any nucleic acid, such as a gene that encodes a polypeptide or protein. Suitable transgenes, for example, for use in gene therapy are well known to those of skill in the art. For example, the vectors described herein can deliver transgenes and uses that include, but are not limited to, those described in U.S. Pat. Nos. 6,547,099; 6,506,559; and 4,766,072; Published U.S. Application No. 20020006664; 20030153519; 20030139363; and published PCT applications of WO 01/68836 and WO 03/010180, and e.g. miRNAs and other transgenes of WO2017/152149; each of which are hereby incorporated herein by reference in their entirety.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A-1F: Fluoroscopic snap shot of swine bile duct distribution after contrast injection via ERCP. Rapid sequence fluoroscopic images (every 3 seconds) during injection of 30 mL of contrast medium at 2 mIJs. This resulted in acinarization of right and subsequently left liver segments without rupture of the bile duct wall (A-F). Therefore, these parameters were deemed optimal for hydrodynamic injection.

FIG. 2A-2B: PCR analysis performed on genomic DNA collected from swine by amplification with primers for the human AKT and NCID gene in swine liver and bile duct. A: Plasmid DNA AKT presence in liver tissue 21 days after ERCP and hydrodynamic injection. At day 21, swine liver were harvested, and DNA was extracted. PCR analysis of 6 samples from each swine liver was performed on genomic DNA by 35 cycles of amplification, denature at 94° C. denature, anneal at 59° C. anneal with primer for human AKT gene. B: Plasmid DNA NICD presence in liver tissue 30 days after ERCP and hydrodynamic injection. At day 30, swine liver were harvested, and DNA was extracted. PCR analysis of 6 samples from each swine liver was performed on genomic DNA by 35 cycles of amplification, denature at 94° C. denature, anneal at 63° C. anneal with primer for human NICD gene. Results show that each location has the plasmid AKT and NICD sequence expression.

FIG. 3A-3C: Western blot shows AKT and Beta-catenin protein expressed in two swine livers. Sixty days after ERCP plasmid hydrodynamic injection, swine (F201 and F202) liver tissue were harvested, lysed and analyzed via western blot. A-B demonstrate successful plasmid DNA integration and transcription into protein. C: Beta-actin functioned as an internal control (C). L (left lobe). C (caudate lobe), R (right lobe), CHD (common hepatic duct), normal pig liver tissue was used as negative control.

FIG. 4A-4F: HA-Tag AKT and MYC-tag beta catenin protein were expressed in swine liver tissue. Swine liver tissue hydrodynamic injected with AKT and beta-catenin plasmids were harvested at day 60, and tissues were analyzed for the expression of plasmid AKT and beta catenin protein via fluorescence microscopy. A-C shows the AKT protein integrated and stably expressed in hepatocytes. D-F shows beta-catenin protein integrated and stably expressed in hepatocytes. This figure demonstrates that hydrodynamically injected plasmids can be stably integrated and expressed in swine hepatocytes (amplification: 20×).

FIG. 5A-5F: HA-Tag AKT and MYC-tag beta catenin protein were expressed in swine hepatocytes and bile duct. Swine liver tissue that underwent hydrodynamic injection with AKT and beta-catenin plasmids were harvested at day 60, and tissues were analyzed for the expression of plasmid AKT and beta catenin protein via fluorescence microscopy. A-C show the anti HA-tag AKT protein inside hepatocytes. D-E demonstrates beta-catenin expressed in bile duct and hepatocytes. F illustrates that some hepatocytes express both AKT and beta-catenin. The figure demonstrate that hydrodynamic injection of plasmids can be stably integrated and expressed in swine hepatocytes and bile duct (amplification: 20×).

FIG. 6A-6B: Gross picture of and H&E staining of swine liver after hydrodynamic injection from common bile duct ex vivo. A: Ex vivo study performed by inserting a 7 Fr biliary stone extraction balloon into the common hepatic duct, inflating the balloon (arrow) and injecting 1% methylene blue diluted in normal saline using an angiographic injector pump. Escalating volumes (10-50 mL) and injection rates (1-4 mUs) were tested. Transient swelling of the entire liver during and immediately after the hydrodynamic injection was observed. The bile duct ruptured when 50 mL of solution was injected at 3 mUs. B: H&E staining shows normal swine liver architecture 60 days after in vivo hydrodynamic gene delivery. Normal liver structure, composed of central vein, portal triads, hepatic sinusoids and liver lobules (amplification: 10×) can be seen.

FIG. 7A-7F: Fluoroscopic snap shot of swine bile duct rupture after inject contrast via ERCP. A-F: Rapid sequence fluoroscopic images (every 3 seconds) during injection of 40 mL of contrast medium at 2 mIJs. D shows rupture of the proximal CHD during injection is represented by contrast extravasation immediately distal to the tip of the balloon catheter just below the hepatic hilum. Therefore, these parameters were too aggressive for hydrodynamic injection.

FIG. 8 shows in vivo intrabiliary pressure through injection of a fluid composition containing nucleic acid with a closed system using a catheter balloon.

FIG. 9A, 9B (includes FIGS. 9B(i) though 9B(iv)), 9C and 9D shows images from Example 2 which follows. FIG. 9A: shows duodenoscope in a biliary anatomy; FIG. 9B (9B(i) though 9B(iv)) shows effects of hydrodynamic injection; FIG. 9C is a further image of significant acinarization throughout the liver; FIG. 9D shows a balloon catheter in the common hepatic duct (above the cystic duct) and in a preferred position for hydrodynamic injection.

FIGS. 10A-10D shows images from Example 4 which follows. FIG. 10A is an endoscopic image with a duodenoscope of the pancreatic orifice. FIG. 11B shows pancreatic cannulation with a sphincterotome preloaded with a guidewire. FIG. 10C is a fluoroscopic image of the guide wire in pancreatic duct. FIG. 10D is a fluoroscopic image with the balloon catheter in pancreatic duct whilst hydrodynamic injection is in process.

### DETAILED DESCRIPTION

An important, but elusive step is the development of a clinical-grade, simple, safe, and efficient in vivo nucleic acid delivery system. Preferred systems may include a non-viral carrier as well as a methodologic approach that would be specific for the liver, minimally invasive, and with the potential to be performed in an outpatient setting.

As discussed, a nucleic acid expression cassette refers to a nucleic acid molecule that may further include one or more transcriptional control elements (such as, but not limited to promoters, enhancers and/or regulatory elements, polyadenylation sequences, and introns) that direct (trans)gene expression in one or more desired cell types, tissues or organs. Typically, they will also contain a transgene, although it is also envisaged that a nucleic acid expression cassette directs expression of an endogenous gene in a cell into which the nucleic acid sequence is inserted.

In certain aspects, a nucleic acid molecule can be used in nucleic acid expression cassettes in conjunction with their natural promoter, as well as with another promoter. For instance, a liver-specific promoter may be used if desired, to increase liver-specificity and/or avoid leakage of expression in other tissues if the target of administration is the subject's liver cells. The liver-specific promoter may or may not be a hepatocyte-specific promoter.

Regulatory sequences also may be used in the nucleic acid expression cassettes. According to a particular embodiment, only one regulatory element is included in the expression cassette. According to an alternative particular embodiment, more than one regulatory element is included in the nucleic acid expression cassette, i.e. they are combined modularly to enhance their regulatory (and/or enhancing) effect. According to a further particular embodiment, two or more copies of the same regulatory element are used in the nucleic acid expression cassette. For instance, 2, 3, 4, or 5 or more copies of a regulatory element may be provided as tandem repeats. According to another further particular embodiment, the more than one regulatory element included in the nucleic acid expression cassette comprises at least two different regulatory elements. In certain embodiments, it is envisaged that the length of the total regulatory element(s) in the nucleic acid expression cassette does not exceed 1000 nucleotides.

The transgene may be homologous or heterologous to the promoter (and/or to the animal, in particular mammal, in which it is introduced, in cases where the nucleic acid expression cassette is used for gene therapy). In addition, the transgene may be a full length cDNA or genomic DNA sequence, or any fragment, subunit or mutant thereof that has at least some biological activity. In particular, the transgene may be a minigene, i.e. a gene sequence lacking part, most or all of its intronic sequences. The transgene thus optionally may contain intron sequences. Optionally, the transgene may be a hybrid nucleic acid sequence, i.e., one constructed from homologous and/or heterologous cDNA and/or genomic DNA fragments. The transgene may also optionally be a mutant of one or more naturally occurring cDNA and/or genomic sequences.

In a particular aspect, a nucleic acid expression cassette does not contain a transgene, but the regulatory element(s) operably linked to the promoter are used to drive expression of an endogenous gene (that thus is equivalent to the transgene in terms of enhanced and/or tissue-specific expression). The nucleic acid expression cassette may be integrated in the genome of the cell or stay episomal.

Other sequences may be incorporated in the nucleic acid expression cassette as well, typically to further increase or stabilize the expression of the transgene product (e.g. introns and/or polyadenylation sequences). Any intron can be utilized in the expression cassettes described herein. The term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of the polyadenylation signal.

The Sleeping Beauty (SB) transposon system has been utilized to promote the integration of transgenes in mammalian cells via a cut-and-paste mechanism.⁵ The system has found its applications mainly in small animals with few in vivo large animal⁶ or human studies.⁷ The system consists of plasmids containing two transcription units, one expressing the enzyme SB transposase and the other expressing the transgene DNA to be inserted into the host genome. In rodents, this technique has resulted in successful expression of coagulation factor IX,⁸ factor VIII,⁹ alpha-1 antitrypsin¹⁰ and many other proteins such that short-term correction of the diseased phenotype was observed. A critical requirement of non-viral gene delivery vehicles, such as SB, is a method to introduce the plasmids in the nucleus of target cells. Several methods have been investigated, mostly in small animals, to deliver non-viral vectors to the liver.¹¹ The most promising, to date, appears to be hydrodynamic injection via a vein¹²⁻²¹. This vascular route, as documented^{22,23}, is technically challenging, time-consuming and by extension expensive, and has, expectedly, cardiovascular side effects. These studies relied on creating relatively high hydrostatic pressure in the vascular bed that promoted plasmid uptake into the target cells.

While the intravascular hydrodynamic injection has been the most commonly utilized route¹²⁻¹⁶, the delivery of plasmids via the bile duct represents an alternative pathway, but has only been evaluated in rodents, through invasive surgical approaches²⁴⁻²⁶. If intra-biliary delivery of plasmids via hydrodynamic administration such as by injection (e.g. endoscopic retrograde cholangiopancreatography (ERCP)-guided) a large animal model could overcome the current challenges faced by intravascular injection, this may promote the commencement of liver targeted gene therapy in humans.

We now describe for the first time efficient hydrodynamic delivery of nucleic acid to liver cells via the biliary tree. The bile duct route has theoretical advantages over intravascular delivery including significantly smaller volume of injection, absence of adverse cardiorespiratory events and reduced risk of systemic toxicity and of systemic dispersal of plasmids, and by extension, increased specificity. Additionally, there is some evidence that bile may contain fewer nucleases versus blood and therefore the intra-biliary route offers the theoretical advantage of improved DNA stability.²⁷

In a specific exemplification, a device-incorporated hydrodynamic delivery of SB and associated plasmids to swine liver was accomplished. Specific devices include a balloon or other space-establishing feature, and particularly exemplified was an ERCP-directed hydrodynamic delivery of nucleic acid to the subject's biliary tree.

In certain preferred systems, pT3-EF 1 a-NICD, pT3-EF 1 a-AKT and pT3-N90-beta-catenin were selected to be delivered as target constructs as they have demonstrated the capability to be integrated into somatic cells in vivo with the guidance of pCMV-SB transposon.²⁸⁻³⁰. In one aspect, it was shown hydrodynamically-mediated non-viral liver gene therapy is simple, effective and safe and useful for human therapy.

In one aspect, it was assessed: (1) optimal parameters for intra-biliary-delivered hydrodynamic gene delivery; (2) demonstrate feasibility of liver cell transduction; and (3) assess whether successful transduction results in stable expression of the delivered plasmid proteins. This invention establishes a minimally invasive method of non-viral gene delivery to the liver.

The injection formulations disclosed herein typically include an effective amount of a nucleic acid expression cassette in a pharmaceutically acceptable carrier suitable for hydrodynamic injection.

The formulations can include a physiologically-acceptable carrier (such as physiological saline or phosphate buffer) selected in accordance with the route of administration and standard pharmaceutical practice.

Pharmaceutical compositions including a nucleic acid expression cassette are prepared according to standard techniques and include a pharmaceutically acceptable carrier. In some embodiments, normal saline is employed as the pharmaceutically acceptable carrier. Other suitable carriers include, e.g., water, buffered water, 0.9% saline, 0.3% glycine, and the like, including glycoproteins for enhanced stability, such as albumin. lipoprotein, globulin, etc. The resulting pharmaceutical preparations can be sterilized by conventional, well known sterilization techniques. The aqueous solutions can then be packaged for use or filtered under aseptic conditions and lyophilized, the lyophilized preparation being combined with a sterile aqueous solution prior to administration. The compositions cab contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The compositions can be administered and taken up into the cells of a subject with or without the aid of a delivery vehicle. For example, nucleic acids may also be delivered by other carriers, including liposomes, polymeric micro- and nanoparticles and polycations such as asialoglycoprotein/polylysine, which can enhance transfection efficiency. In some embodiments, the composition is incorporated into or encapsulated by a nanoparticle, microparticle, micelle, synthetic lipoprotein particle, or carbon nanotube. Preferred carriers include targeted liposomes (Liu, et al. Curr. Med. Chem., 10: 1307-1315 (2003)) such as immunoliposomes, which can incorporate acylated mAbs into the lipid bilayer. Polycations such as asialoglycoprotein/polylysine may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA for transfer.

Typically the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

Typically, the formulations include an amount of a nucleic acid expression cassette effective to modify the genome of one or more targets cells in a subject following hydrodynamic administration of the formulation to the subject. In preferred embodiments, the amount is effective to modify the genome of enough of the target cells to treat, reduce, or prevent one or symptoms a disease being treated, or to produce an alteration in a physiological or biochemical manifestation thereof.

Preferred dosage amounts also can be determined empirically as well as upon consideration of for example the therapeutic context and desired result age, and general health of the recipient.

The examples which follow also demonstrate effective dosages. Additionally, Khorsandi, et al, Cancer Gene Therapy, 15:225-230 (2008) reported administering pigs dosages of between 10 mg and 20 mg of plasmid, and humans dosages of 1 mg to 45 mg of plasmid by selective hydrodynamic injection (regional circulation), and all dosages were found to be safe and tolerated by the subjects. Therefore, generally, the dosages can range from about 0.001 mg to about 1,000 mg, more preferable about 0.01 mg to about 100 mg of nucleic acid expression cassette genome editing composition, depending on the subject to be treated, the route of administration, the targets cells.

Hydrodynamic injection, also referred to as high pressure injection, is a method of administering nucleic acids in vivo. Hydrodynamic injection is amenable to delivery of “naked” nucleic acids, and therefore does not require viral carriers that can require laborious procedures for preparation and purification, and carry with them concerns about the possibility for recombination with endogenous virus to produce a deleteriously infectious form. Hydrodynamic injection also does not appear to cause the immune response and other side effects that render the repeated administration of viral vectors problematic. Being different from carrier-based strategy and the earlier work employing hypertonic solution and elevated hydrostatic pressure to facilitate intracellular DNA transfer, hydrodynamic gene delivery relies on hydrodynamic pressure generated by a rapid injection of a large volume of fluid to deliver genetic materials into parenchyma cells. See also Suda and Liu, et al, Molecular Therapy, 15(12):2063-2069 (2007), Al-Dosari, et al, Adv. Genet. 54: 65-82 (2005). Kobayashi, et al, Adv Drug Deliv. Rev. 57: 713-731 (2005), and Herweijer and Wolff. Gene Ther. 14: 99-107 (2007).

Injection volume and injection speed can also be important consideration in the efficacy of hydrodynamic delivery. Faster injection speeds are generally preferred.

The solution can be delivered by any means suitable for delivering the desired volume at the desired rate. For example, the solution can be administered using an injection device such as a catheter, syringe needle, cannula, stylet, balloon catheter, multiple balloon catheter, single lumen catheter, and multilumen catheter. Single and multi-port injectors may be used, as well as single or multi-balloon catheters and single and multilumen injection devices. A catheter can be inserted at a distant site and threaded through the lumen of a vein so that it resides in or near a target tissue. The injection can also be performed using a needle that traverses the skin and enters the lumen of a vessel.

Administration can be aided by the incorporation of pump or other system to facilitate delivery of the desired volume at the desired pressure. In a particular embodiment, administration includes use of a computer-assisted system enabling real-time control of the injection based on the hydrodynamic pressure at the injection site of the tissue. Precise control of injection can avoid tissue damage caused by too heavy an injection, or low gene delivery efficiency due to insufficient volume or injection speed.

FIG. 8 shows in vivo intrabiliary pressure through injection into a pig subject of a fluid composition containing nucleic acid with a closed system using a catheter balloon. As shown in FIG. 8, intrabiliary pressure increases sharply from a baseline upon commencing injection but then plateaus at an elevated level (about 150 mmHG) during the injection period. Upon terminating the injection (shown as “Injection Concludes”) in FIG. 8, the intrabiliary pressure decreases but remains for some time at a higher level than the pre-injection intrabiliary pressure, at least until the extraction balloon (used to create a substantially closed space) is deflated.

For instance, in certain preferred system, upon injection concluding, the intrabiliary pressure may remain at 1, 2, 3, 4, 5, 6, 7, 8, 9, 10, 12, 14, 15, 16, 18 or 20 mmHG or more higher than the pre-injection intrabiliary pressure, at least until the extraction balloon is deflated.

Gene delivery can also be optimized and toxicity (tissue damage) minimized by varying the volume of the solution and the speed of injection; varying the osmotic pressure by the addition of mannitol to the injection solution; increasing fluid and DNA extravasation, e.g., by vessel dilation using papaverine, hyaluronidase, or VEGF protein pre-injection, and the like.

In some embodiments one or more vessels or ducts are occluded to reduce or prevent flow of the solution in one or more directions, for example, back flow. Methods of occluding ducts or vessels can be accomplished by varying methods. For example, the injection apparatus itself can reduce back flow. In some embodiments one or more cuffs, tourniquets or combination thereof is used to reduce or prevent solution flow in one or more directions. The cuff or tourniquets can be applied directly to the vessel, or to the tissue surrounding the vessel. In some embodiments, one or more balloon catheters is used to reduce or prevent solution flow in one or more directions. For at least certain systems, use of a balloon catheter to create a substantially closed system may be preferred.

The occlusion(s) can be carried out using non-invasive procedures, minimally invasive procedures, or invasive procedures. For example, in some embodiment, the ducts vessels are occluded by an open surgical procedure. In other embodiments, the ducts or vessels are occluded using a minimally invasive procedure such as percutaneous surgery. Varying approaches can be carried out through the skin or through a body cavity or anatomical opening and may incorporate the use of catheters, arthroscopic devices, laparoscopic devices, and the like, and remote-control manipulation of instruments with indirect observation of the surgical field through an endoscope or large scale display panel, etc.

The disclosed hydrodynamic delivery methods are preferably via the biliary tree of a subject and do not involve delivery through the blood vessels such as an artery or a vein.

The target cells, and therefore the particular method of hydrodynamic injection, are typically selected based on disease to be treated. In some embodiments, the target cells are liver cells, kidney cells, or pancreatic cells.

In preferred embodiments, the target cells are parenchymal cells. Parenchymal cells are the distinguishing cells of a gland or organ contained in and supportcd by the connective tissue framework. The parenchymal cells typically perform a function that is unique to the particular organ. The term “parenchymal” often excludes cells that are common to many organs and tissues such as fibroblasts and endothelial cells within blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells.

Applications of the disclosed compositions and methods include gene therapy. e.g., to treat a disease, or as an antiviral, antipathogenic, or anticancer therapeutic. The disclosed methods can be used to treat any disease or condition wherein genome modification of target cells is effective to treat the disease or condition, and wherein the target cells can be transfected with the disclosed compositions by hydrodynamic injection.

Preferably the cells leading to the disease pathology can be transfected by hydrodynamic injection. Preferred target cells include those discussed above, including liver cells, kidney cells and pancreatic cells.

### Catheter Delivery Systems Such as to Deliver to Biliary Tree/Liver, Pancreatic Duct/Pancreas/Kidney

In preferred systems, such as to access and deliver therapeutic agents to the biliary tree/liver, pancreatic duct/pancreas, renal collecting system/kidney, a catheter delivery system suitably may be detectable, for example fluoroscopically visible. In an administration protocol, a catheter suitably may be placed in position for administration with or without a guidewire. If used, guidewire of varying configurations may be suitably used, include those having a inner diameter of from about 0.01 to 0.04 inches for example diameters of 0.018 inch, 0.025 inch, 0.035 inch or other diameters. The guidewire suitably can be radio-opaque if desired. Preferred catheters may have one or a plurality of ports of equal or varying diameters, for example 2, 3, 4 or more ports. The largest diameter port would be for the hydrodymaic delivery. Preferred catheters suitably can be 6 Fr-12 Fr in size as well as other sizes.

If two or more ports are used, then one port can be used to inflate the occlusion balloon or activate a mechanism to occlude the lumen and prevent/decrease the risk of catheter migration during injection and the other port will allow for the guidewire initially to run through it but then the guidewire can be removed and contrast/hydrodynamic delivery of a solution can be performed through that same port.

If three ports are used, a preferred configuration can include a first port for the occlusion balloon etc, a second distinct port for the guidewire/contrast/hydrodynamic delivery, and a third distinct port for the pressure catheter.

If four ports are used, a preferred configuration can include one for the occlusion balloon etc, one for the guidewire/contrast, one for contrast/hydrodynamic delivery, one for the pressure catheter.

In certain preferred aspects, the catheter may have the pressure catheter embedded in its tip so that only 2-3 additional ports are required.

If two additional ports are utilized, one can be used for the occlusion balloon etc, and a second can be used for the guidewire/contrast/hydrodynamic delivery. If three additional ports are utilized, one can be used for the occlusion balloon etc, a second can be used for the guidewire/contrast, and a third can be used for the contrast/hydrodynamic delivery.

In additional preferred systems, in addition to the catheter, a mechanism can be utilized of injecting the solution at a variety of pressures, volumes, duration, flow rates that may be fixed or titrated to the feedback from the pressure sensor

### Biliary Hydrodynamic Injection

For particularly preferred biliary hydrodynamic injection of therapeutic materials (e.g. nucleic acid expression cassette), the systems will be suitable for both male and female patients. In a preferred protocol, an endoscope or echoendoscope may be used, and a catheter is placed via the transoral route/mouth into the bile duct either through the major papilla or via direct puncture through the duodenal and advanced to the upstream biliary tree in a retrograde fashion. A balloon or any mechanism can be used to occlude the lumen is inflated/activated and the catheter is withdrawn (or kept in position) to occlude one of the common hepatic duct, left hepatic duct, right hepatic duct. The purpose of the balloon/other mechanism is to not only occlude the lumen and prevent antegrade flow of the solution injected but also to anchor the catheter in position. The catheter suitably can be placed into the above position under direct visualization (cholangioscopy) and/or under fluoroscopic visualization. If fluoroscopy is used, the biliary tree could opacify and this would aid in positioning the catheter tip in the area of interest. A guidewire suitable can be used to aid in the advancement of the catheter to the optimal position. An optimal position could be the common hepatic duct to allow for the solution injected to enter the entire biliary system of the liver. An optimal position also could be one of the hepatic ducs (left hepatic duct, right hepatic duct) such that only a portion of the hepatic parenchyma/hepatocytes is subject to hydrodynamic injection. Bile and or contrast agent if used can be aspirated to confirm the position in the biliary tree and to remove as much bile as possible to minimize retrograde reflux of bile into the hepatic parenchyma/hepatocytes. Suitably, the biliary tree is lavaged with a solution such as saline (but could be other solutions) to remove as much bile as possible to minimize retrograde efflux of bile into the hepatic parenchyma/hepatocytes. Suitably, the biliary tree is primed with the solution of interest. Suitably, the balloon could be inflated at this point (prior to the hydrodynamic injection) or at any point prior to the catheter tip being in position. Hydrodynamic injection is then suitably performed preferred such that the solution of interest enters the various cells of hepatic parenchyma/hepatocytes. The injection suitably may be performed under fluoroscopic guidance. The injection may or may not be performed with a power injector. The volume of solution injected, the speed of injection, the duration of injection, and the pressure of injection could be detected in real time by a pressure sensor incorporated into the catheter or running through the working channel of the catheter. The pressure sensor maybe connected to a system which is able to regulate the injection parameters such that a variety of pressure waveforms could be generated (eg. initial high pressure followed by stable moderate pressure, or a stable pressure throughout OR multiple bursts of high pressure on a background of moderate pressure (sawtooth pattern)) prior to returning the baseline when the injection stops and/or the balloon/occluding mechanism is deflated. Suitably, contrast in reinjected to opacify the biliary tree to assess for leak. This can be performed by re-inflating the balloon or not using the balloon at all. The catheter is then suitably removed.

The method recognizes that the system is not an entirely “closed” system in the absolute sense as a significantly greater volume can be injected into the biliary tree than what the biliary tree could otherwise hold. The solution therefore likely/almost certainly does enter the branches of the hepatic sinusoids, portal vein and hepatic vein and this could result in the system spread of the solution.

The method may or may not be performed with prophylactic and/or post procedure antibiotics injected intravenously or lavaged into the biliary tree.

Suitably, the flow rate of the injection could be up to or greater than 2 mL/sec, 3 ml/sec, 5 ml/s, 10 ml/sec, or other value.

Suitably, the pressure in the biliary during hydrodynamic gene delivery could be up to or greater than 40 mmHg, 50 mmHg or more. In at least some aspects, an upper limit may be 200 mmHg, although in certain systems higher pressures may be employed.

The delivery injected suitably may have various concentration of the substance of interest (gene/DNA etc) and the solution could have various viscosity.

Hydrodynamic injection could occur using various volumes of solution, for example 20 mL, 30 mL, 40 mL, 60 mL, 80 mL, 100 mL, 120 mL, 150 mL, 180 mL, 200 mL or other amount.

The balloon suitably can be kept inflated for just the duration of injection or for an extended period of time after the hydrodynamic injection/power injection is completed

The injection suitably can be repeated at the same site or at other sites in the biliary tree during the same procedure. For example the other hepatic duct or at the same site to optimize transfection

The entire process of hydrodynamic injection can be repeated one or more times to the same site or new sites in the biliary tree

### Pancreatic Hydrodynamic Injection:

In preferred systems, such as to access and deliver therapeutic agents to the biliary tree/liver, pancreatic duct/pancreas, renal collecting system/kidney, suitably patients may be male or female mammals such as humans. In preferred aspects, an endoscope or echoendoscope or other device may be used and a catheter suitably placed via the transoral route/mouth into the pancreatic duct either through the major papilla or via direct puncture through the duodenal/gastric wall and advanced to the upstream biliary tree in a retrograde or antegrade fashion. A balloon or other mechanism suitably can be used to occlude the lumen is inflated/activated and the catheter is withdrawn (or kept in position) to occlude one of the main pancreatic duct. The purpose of the balloon/other mechanism is to not only occlude the lumen and prevent antegrade flow of the solution injected but also to anchor the catheter in position.

Suitably, a catheter can be placed into the above position under direct visualization (pancreatoscopy) and/or under fluoroscopic visualization

If fluoroscopy is used, the pancreatic tree could opacify and this would aid in positioning the catheter tip in the area of interest

A guidewire suitably can be used to aid in the advancement of the catheter to the optimal position

In certain aspects, an optimal position could be the main pancreatic duct to allow for the solution injected to enter the entire pancreatic parenchyma

In additional aspects, an optimal position could be upstream from the entry of the accessory pancreatic duct such that the is no or minimal leakage of solution/pressure through the accessory pancreatic duct and minor papilla.

Suitably, if pancreatic ductal fluid and or contrast agent if used is aspirated to confirm the position in the pancreatic tree and to remove as much pancreatic fluid as possible to minimize retrograde reflux of pancreatic fluid into the pancreatic parenchyma,

Suitably, the pancreatic parenchmya includes acinar cells and islet cells. In certain aspects, acinar cells are treated.

In preferred applications, the pancreatic tree is lavaged with a solution such as saline (but could be other solutions) to remove pancreatic fluid to minimize retrograde efflux of pancreatic fluid into the pancreatic parenchyma.

Again, in preferred protocols, the pancreatic tree is primed with the solution of interest. The balloon suitably could be inflated at this point (prior to the hydrodynamic injection) or at any point prior to the catheter tip being in position.

Hydrodynamic injection is then suitably performed such that the solution of interest enters the various cells of the pancreatic parenchyma. The injection may be performed under fluoroscopic guidance. The injection may or may not be performed with a power injector.

The volume of solution injected, the speed of injection, the duration of injection, and the pressure of injection could be detected in real time by a pressure sensor incorporated into the catheter or running through the working channel of the catheter. The pressure sensor suitably may be connected to a system which is able to regulate the injection parameters such that a variety of pressure waveforms could be generated (e.g. initial high pressure followed by stable moderate pressure, or a stable pressure throughout OR multiple bursts of high pressure on a background of moderate pressure (e.g. sawtooth pattern)) prior to returning the baseline when the injection stops and/or the balloon/occluding mechanism is deflated. Contrast suitably may be reinjected to opacify the pancreatic tree to assess for leak. This can be performed by re-inflating the balloon or not using the balloon at all. The catheter is then suitably removed.

The method recognizes that the system may not be entirely “closed” system in the absolute sense as a significantly greater volume can be injected into the pancreatic tree than what the pancreatic tree could otherwise hold. The solution therefore likely/almost certainly does enter the branches of the superior mesenteric vein, splenic vein, superior and inferior pancreatoduodenal veins and this could result in the system spread of the solution

The method may or may not be performed with prophylactic and/or post procedure antibiotics injected intravenously or lavaged into the pancreatic tree

The flow rate of the injection suitably can vary for example up to or greater than 2 mLJsec, 3 ml/sec, 5 ml/s, 10 ml/sec or other. The pressure in the pancreatic duct during hydrodynamic gene delivery also may vary and suitably may be up to or greater than 40 mmHg, 50 mmHg or other. In certain systems, an upper limit may be 200 mmHg, although higher pressures may be useful in certain systems.

The solution injected suitably can have various concentration of the substance of interest (gene/DNA etc) and the solution could have various viscosity

Hydrodynamic injection suitably can use various volumes of solution, for example up to or greater than 20 mL, 30 mL, 40 mL, 60 mL, 80 mL, 100 mL, 120 mL, 150 mL, 180 mL, 200 mL or other amount.

In certain aspects, the balloon can be kept inflated for just the duration of injection or for an extended period of time after the hydrodynamic injection/power injection is completed

In certain aspects, the injection can be repeated at the same site or at other sites in the pancreatic duct during the same procedure to optimize transfection

In additional aspects, the entire process of hydrodynamic injection can be repeated one or more times to the same site or new sites in the pancreatic ductal system

### Renal Hydrodynamic Injection:

In preferred systems of renal hydrodynamic injection, suitably patients may be male or female mammals such as humans.

In a preferred protocol, preferably under sterile technique, a catheter may be placed via the urethra into the ureter (either the left or right) and advanced to the renal pelvis. A balloon or other mechanism suitably can be used to occlude the lumen is inflated/activated and the catheter is withdrawn (or kept in position) to occlude one of the major calyx, the renal hilus, the renal pelvis, or the proximal aspect of the ureter. The purpose of the balloon/other mechanism is to not only occlude the lumen and prevent antegrade flow of the solution injected but also to ancho the catheter in position.

Suitably, the catheter can be placed into the above position under direct visualization (ureteroscopy) and/or under fluoroscopic visualization

Suitably, if fluoroscopy is used, the renal collecting system could opacify and this would aid in positioning the catheter tip in the area of interest

A guidewire suitably can be used to aid in the advancement of the catheter to the optimal position

An optimal position could be the renal pelvis, renal hilum, or proximal ureter to allow for the solution injected to enter the entire collecting system of that kidney

An optimal position also could be one of the major calyx such that only a portion of the renal parenchyma is subject to hydrodynamic injection

If urine and or contrast agent if used is aspirated to confirm the position in the renal collecting system and to remove urine to minimize retrograde efflux of urine into the renal parenchyma

Suitably, the collecting system is lavaged with a solution such as saline (but could be other solutions) to remove as much urine as possible to minimize retrograde efflux of urine into the renal parenchyma.

The renal collecting system preferably is primed with the solution of interest. The balloon if used could be inflated at this point (prior to the hydrodynamic injection) or at any point prior to the catheter tip being in position.

Hydrodynamic injection is then suitably performed such that the solution of interest enters the various cells of renal parenchyma. The injection may be suitably performed under fluoroscopic guidance. The injection may or may not be performed with a power injector.

The volume of solution injected, the speed of injection, the duration of injection, and the pressure of injection could be detected in real time by a pressure sensor incorporated into the catheter or running through the working channel of the catheter.

The pressure sensor if used suitably may be connected to a system which is able to regulate the injection parameters such that a variety of pressure waveforms could be generated (e.g. initial high pressure followed by stable moderate pressure, or a stable pressure throughout or multiple bursts of high pressure on a background of moderate pressure (e.g. sawtooth pattern)) prior to returning the baseline when the injection stops and/or the balloon/occluding mechanism is deflated.

In certain systems, contrast may be reinjected to opacify the collecting system to assess for leak. This can be performed by re-inflating the balloon or not using the balloon at all

The catheter is then suitably removed.

The method recognizes that the system is not an entirely “closed” system in the absolute sense as a significantly greater volume can be injected into the collecting system than what the collecting system could otherwise hold. The solution therefore likely/almost certainly does enter the branches of the renal artery and vein and this could result in the system spread of the solution

The method may or may not be performed with prophylactic and/or post procedure antibiotics injected intravenously or lavaged into the collecting system

The flow rate of the injection suitably may be up to or greater than 2 mL/sec, 3 ml/sec, 5 ml/s, 10 ml/sec or other amount.

The pressure in the collection system during hydrodynamic gene delivery suitably may be up to or greater than 40 mmHg, 50 mmHg or other amount. In certain systems an upper pressure limit may be about 200 mmHg, although in certain systems higher pressure may be suitably employed.

The solution injected suitably may have various concentration of the substance of interest (gene/DNA etc) and the solution could have various viscosity

Hydrodynamic injection could occur using various volumes of solution, for example up to or greater than 20 mL, 30 mL, 40 mL, 60 mL, 80 mL, 100 mL, 120 mL, 150 mL, 180 mL, 200 mL or other amount.

The balloon if used suitably can be kept inflated for just the duration of injection or for an extended period of time after the hydrodynamic injection/power injection is completed

The injection suitably can be repeated at the same site or at other sites in the renal collecting system during the same procedure. For example, at another major calyx, at the other kidney, or at the same site to optimize transfection.

The entire process of hydrodynamic injection suitably can be repeated one or more times to the same site or new sites in the collecting system.

The following non-limiting examples are illustrative.

### Example 1

Animal and Study Conditions.

A total of 15 Yorkshire pigs (Sus scrofa domestica) weighing 40-50 kg and aged four to six months at study initiation were obtained from a commercial, closed herd swine vendor (Archer Farms, Darlington, Md.). Environmental acclimation at 72° F.±2° F., 30-70% relative humidity, 14 hr:10 hr (light: dark cycle) and approximately 15 air changes/hour occurred for one week prior to study initiation. Swine were housed individually in 24 ft² indoor runs and fed Teklad Mini-swine diet (No. 8753, Harlan Tekland, Madison, Wis.). Water was provided ad libitum prior to study initiation. All experimental animal procedures were approved by the Institutional Animal Care and Use Committee at the Johns Hopkins University and conducted in compliance with the Animal Welfare Act, applicable Animal Welfare Regulations, and the Guide for the Care and Use of Laboratory Animals at an AAALAC-accredited facility.³¹-3³

Determination of Maximal Tolerable Injection Parameters of the Swine Bile Duct. Initially, the inventors performed ex vivo experiments using standard endoscopic accessories on three swine livers to determine the maximal tolerated injection parameters necessary to rupture the biliary tree. These ex vivo studies were utilized to inform parameters for initial in vivo non-survival studies on three swine. After the swine were anesthetized, the duodenoscope (ED-3490 TK, Pentax, Montvale, N.J.) was inserted through the mouth and positioned in the proximal duodenum in front of the biliary orifice. The common bile duct (CBD) was cannulated with an extraction balloon preloaded with a 0.035 inch hydrophilic guidewire (Dreamwire, Boston Scientific, Natick, Mass.). Selective biliary cannulation was technically simple and safe (no risk of pancreatitis) as the opening of the pancreatic duct is separate in swine. Under fluoroscopic guidance (Allura Xper FD20, Philips Medical Systems N. A., Bothell, Wash.), 3 mL boluses of one third strength iohexol contrast medium (Omnipaque, 350 mg/mL, GE Health Co., Princeton, N.J.) were injected into the biliary tree to opacify important landmarks (cystic duct, hepatic hilum and main intrahepatic ducts). The guidewire was then inserted into the intrahepatic ducts, and an extraction balloon was advanced to the common hepatic duct (CHD). The extraction balloon was subsequently advanced to the CHD as the injection from this location would allow the entire liver to be exposed to the injected solution. At this point, the balloon was inflated with air to 12 mm such that it was wedged against the duct wall. The guidewire was removed and the angiographic injector apparatus was connected to the guidewire port of the extraction balloon. Under fluoroscopy, X mL injection volumes (X=10, 20, 30, 40) of one third strength iohexol contrast medium were injected at Y mUs injection rate (Y=1, 2, 3) with the maximal pressure set to 999 psi. The balloon remained inflated for 30 seconds after completion of each injection. Injection parameters were sequentially tested at 10-minute intervals in ascending order until the rupture of the bile duct as evidenced by extravasation of contrast medium. The duodenoscope was then withdrawn, and the swine euthanized.

Hydrodynamic Gene Delivery to Effectuate Plasmid Transduction.

The preparation of the pigs (n=12) and the instruments utilized were similar to the aforementioned experiments. The bile duct was cannulated, 3 mL of contrast medium injected and the balloon advanced over the guidewire into the CHD. The extraction balloon was inflated to 12 mm and the guidewire removed. The guidewire port was primed with 1.5 mL of plasmids. Then 30 mL of plasmids was injected at 2 mUs (based on the experiment above) with the maximal pressure set to 999 psi. The balloon remained inflated for 2 minutes. After balloon deflation, to confirm the integrity of the biliary tree, a balloon occlusion cholangiogram was performed by withdrawing the extraction balloon to the distal CBD, inflating the balloon to 12 mm, and injecting 5 mL of contrast medium. The duodenoscope was then withdrawn and swine recovered under veterinary supervision until supine. The clinical health of the animals was assessed at least once daily over the duration of the study.

Plasmids and their Allocation.

For the purposes of stable gene expression in target liver of swine, the inventors chose target plasmids combined with Sleeping Beauty-mediated somatic integration. The constructs pT3-EFla-NICD, pT3-EFla-AKT, pT3-N90-beta-catenin, and transposon plasmid pCMV-SB were used. The allocation of the constructs into each of the three groups are shown in Table 1. Note, 8 swine received a single plasmid (AKT or NICD) and 4 swine received a combination of 2 plasmids (AKT and beta-catenin) (Table 1). Transduction of plasmids to each of the 12 swine liver were assessed via PCR. Protein expression was assessed in the 4 swine that received the combination of 2 plasmids and were survived to 60 days. Protein expression was illustrated by Western blot and the location of integrated gene protein expression was determined by immunofluorence staining.

TABLE 1

| Swine | Plasmids combination | Survival time |
|---|---|---|
| 4 | PT3-EF1a-AKT + PCMV- | 21 days |
| | Sleeping beauty | |
| 4 | PT3-EF1a-NICD + PCMV- | 30 days |
| | Sleeping beauty | |
| 4 | PT3-EF1a-AKT and PT3-N90- | 60 days |
| | beta-catenin + PCMV-Sleeping | |
| | Beauty | |

Hydrodynamic Injection Parameters.

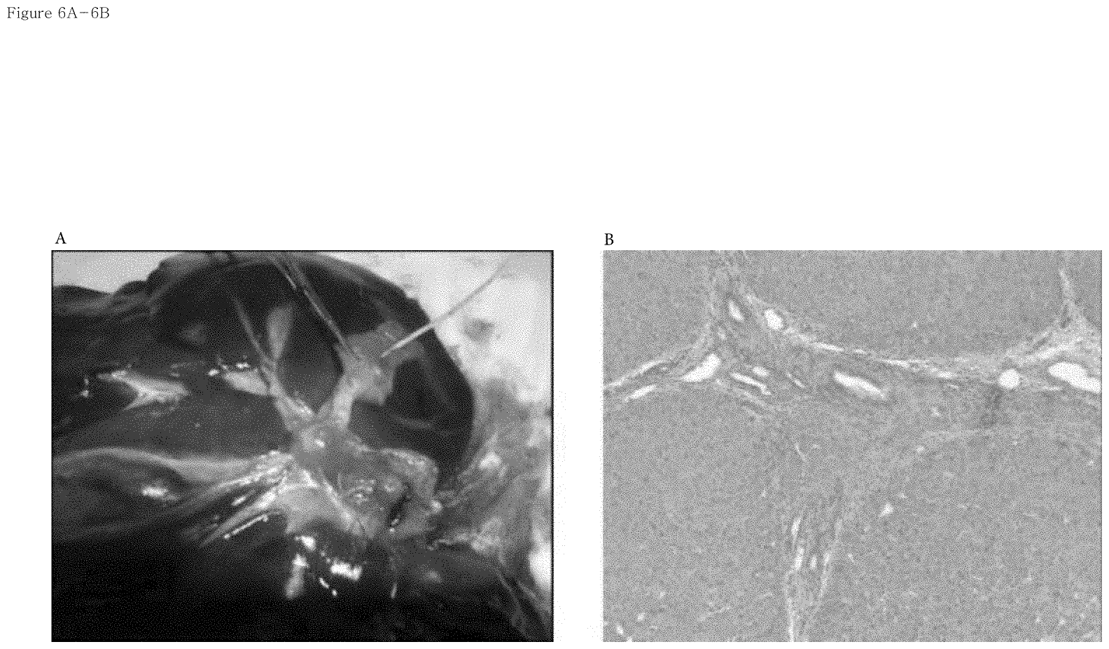

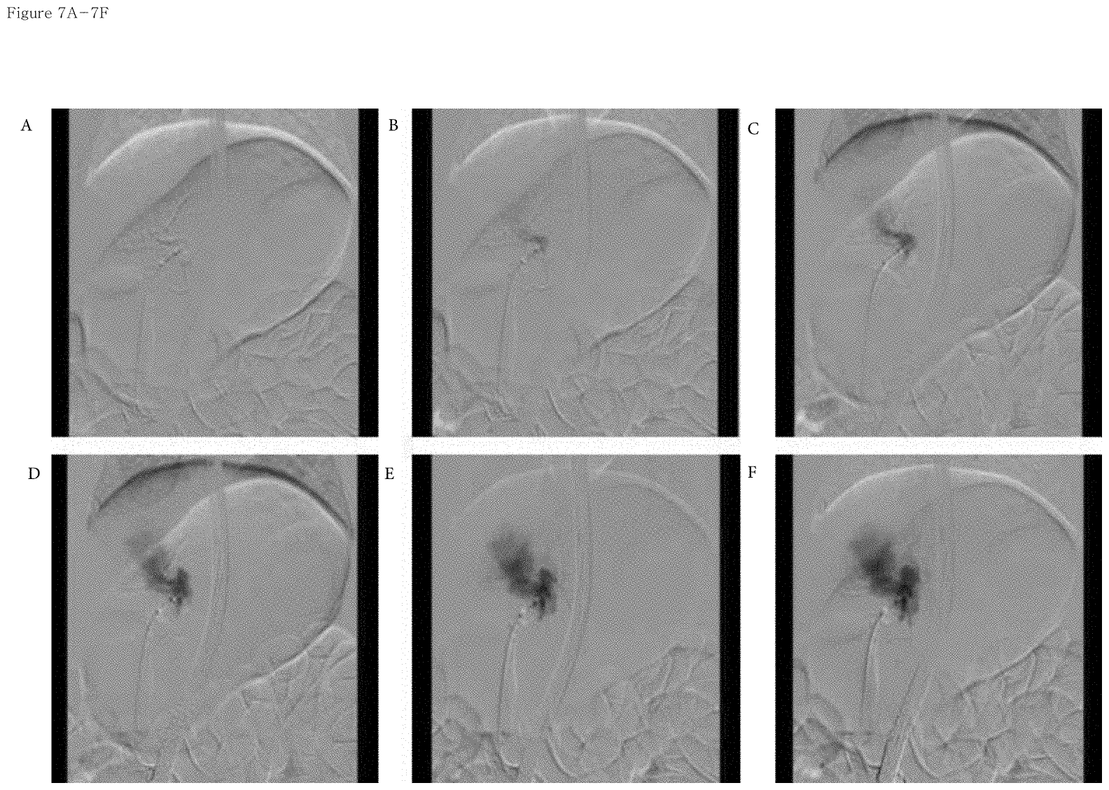

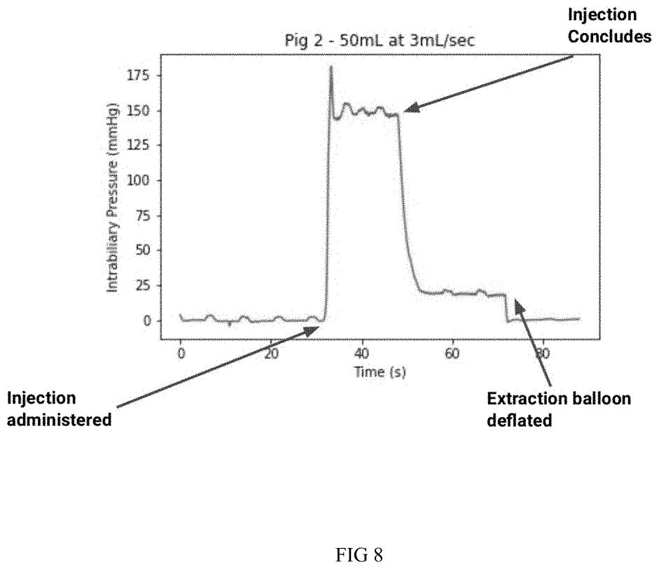

Initial experiments were performed ex vivo with the specific purpose of clarifying the upper limits of safe injection speed and volumes. The inventors injected normal saline through the catheter and noted transient swelling of the liver during and immediately after the hydrodynamic injection. Experiments revealed that the bile duct ruptured when 50 mL of solution was injected at a speed of 3 mUs. In each of the livers, rupture occurred immediately distal to the tip of the balloon just below the hepatic hilum (FIG. 6).

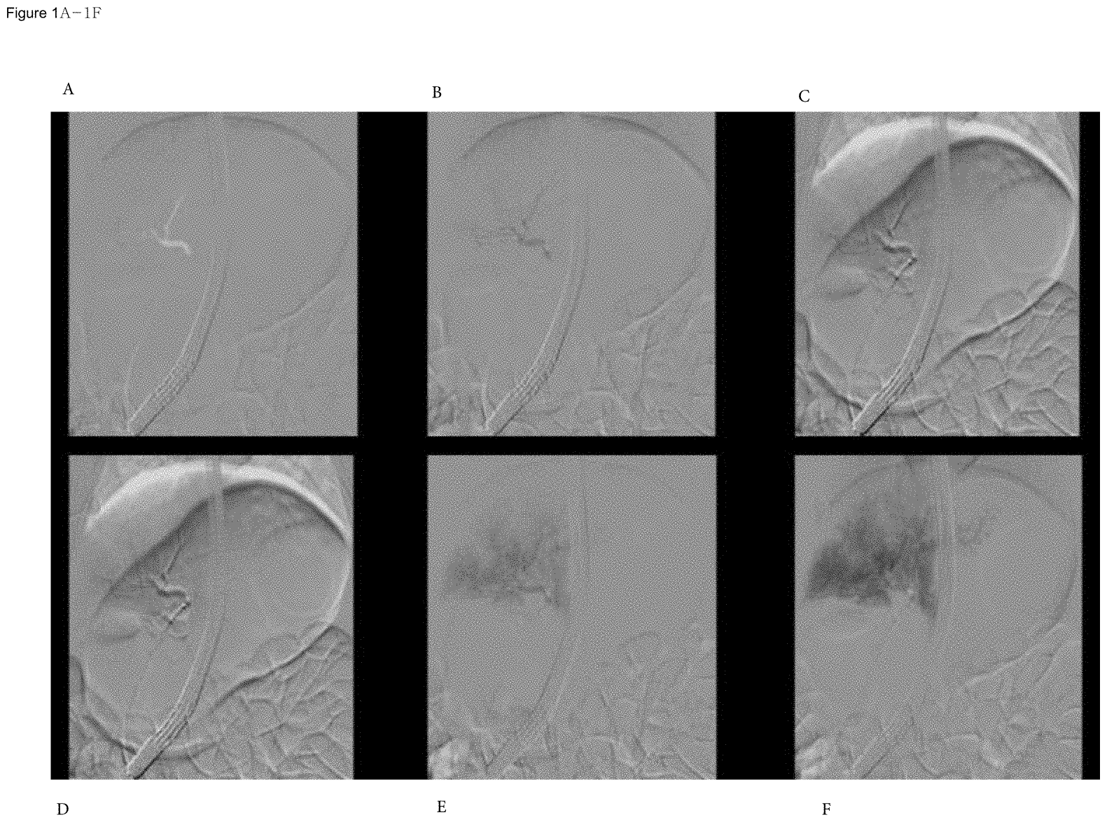

The segmental anatomy of the swine liver is similar to that of a human with regard to vascularity and the biliary tree distribution.³⁴ The liver contains six lobes including the quadrate, caudate, right medial and lateral, left medial and lateral. The common hepatic duct (CHD) enters the liver caudal and dorsal to the gallbladder. The CHD is found in the porta hepaticus, ventral to the portal vein and the hepatic artery.³⁴ The inventors found the biliary orifice located approximately 2 cm distal to the pylorus on the posterior inferior wall.³⁴ For all ERCP procedures, the duodenoscope was placed in the short position enface to the biliary orifice. In vivo experiments revealed that cannulation of the common bile duct (CBD) and intrahepatic ducts was technically simple. Contrast injection demonstrated that the diameters of CBD was 6-8 mm, CHD 3-5 mm and the main intrahepatic ducts 2 mm, respectively. Despite injecting volumes as high as 20 mL at rates of 4 mUs, no contrast medium escaped around the balloon into the CBD during hydrodynamic injection. This indicated that an adequate seal was created to facilitate the generation of hydrostatic pressure. Injecting volumes greater or equal to 20 mL, regardless of rate, demonstrated acinarization of the liver parenchyma indicating that contrast had exited the biliary tree. Injecting 40 mL at 2 mIJs resulted in rupture of the proximal CHD during the process of injection (FIG. 7). Injecting 30 mL at 2 mUs resulted in acinarization of all liver segments without rupture of the bile duct wall (FIG. 1). For the following experiments the inventors have used these parameters and encountered no rupture or other local complications.

Tolerability of Hydrodynamic Injection.

Intravascular delivery of hydrodynamic therapy in animals is associated with a high rate of cardiovascular stress. Establishing a novel method to deliver hydrodynamic therapy that would be void of any cardiovascular complications was one of the main motivations for the current invention. Therefore, the inventors carefully monitored for any systemic, cardiovascular or respiratory complications. Through these experiments, the inventors demonstrated that there were no changes in physiological parameters observed during or after the injection, including electrocardiogram, heart rate, respiration rate, temperature, blood pressure, and oxygen saturation. There were no intra- or post procedural adverse events observed. The animals were alert, responsive, defecating, urinating, drinking, and eating well in after the procedure and for the following days. Animals did not exhibit signs of sepsis, peritonism or jaundice. Labs, including liver function tests and complete blood count demonstrated values within normal limits at 7 days and at the time of sacrifice (day 21, 30 or 60) (data not shown). These results appear to suggest that the process of ERCP and intra-biliary hydrodynamic gene delivery has no significant negative physiological or functional impact.

Impact of Hydrodynamic Injection on Hepatic Tissue.

The balloon occlusion cholangiogram on the day of sacrifice revealed mild diffuse dilation of the extra- and intrahepatic biliary tree by approximately 25%. There was no focal dilation or stricture of the bile ducts. At necropsy, gross examination of the abdomens were within normal limits. There was no ascites and the liver surface had a normal appearance. The liver was sectioned in 1 cm thick slices with normal hepatic parenchyma observed. Microscopic examination revealed no evidence of chronic liver injury or accumulation of lymphocytes. There was no enlargement of hepatic sinusoids (FIG. 6). These results suggest that hydrodynamic gene delivery via the biliary tree is safe. It is unclear if the observed mild dilation in the biliary tree is specific to swine or likely to occur in humans. Furthermore, even if it were to occur, it is questionable that there would be any consequence as a result of the mild dilation. Of note, dilation in the common bile duct is sometimes observed after cholecystectomy with no known negative consequences.

Plasmid DNA can Successfully be Transferred into Swine Liver Hepatocytes.

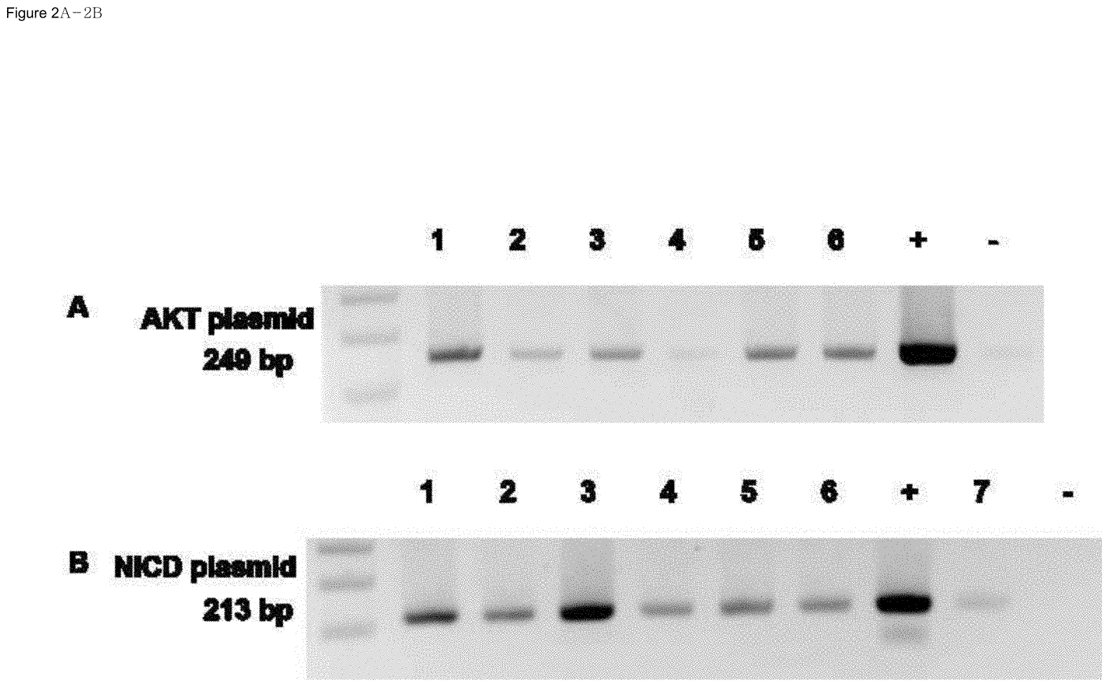

Swine livers specimens (6 from each swine) from day 21 and day 30, respectively, post hydrodynamic injections were harvested and DNA was extracted (FIGS. 2A and 2B). The 249 base pair (bp) PCR product of AKT and 213 bp product of NICD, respectively, were detected in the left (proximal and distal), right (proximal and distal), caudate liver lobe and CHD in each of the 8 swine at day 21 and 30, respectively. Therefore, based on PCR data, these two groups of experiments demonstrated that single genes (AKT or NICD) can be successfully transduced into liver tissue. Considering these initial positive results, the inventors then wanted to assess whether a combination of two plasmids can also be delivered. To this end, AKT+beta-catenin in 1:1 ratio were delivered together with pCMV-SB at the same ratio of 25:1 gene DNA:SB DNA. Thus, another four swine underwent ERCP and hydrodynamic injection and were survived for 60 days. Both AKT and beta-catenin plasmid DNA were detected after 60 days in each of the swine throughout all liver lobes as well as in CHD (data not shown). These results demonstrate hydrodynamic injection of PT3-EF 1 a-AKT and PT3-N90-beta-catenin can successfully transduce hepatocytes and the presence after 2 months indicates stable integration of the constructs in the hepatocyte genomic DNA.

Protein Expression of the Delivered Genes in Swine Liver Tissue.

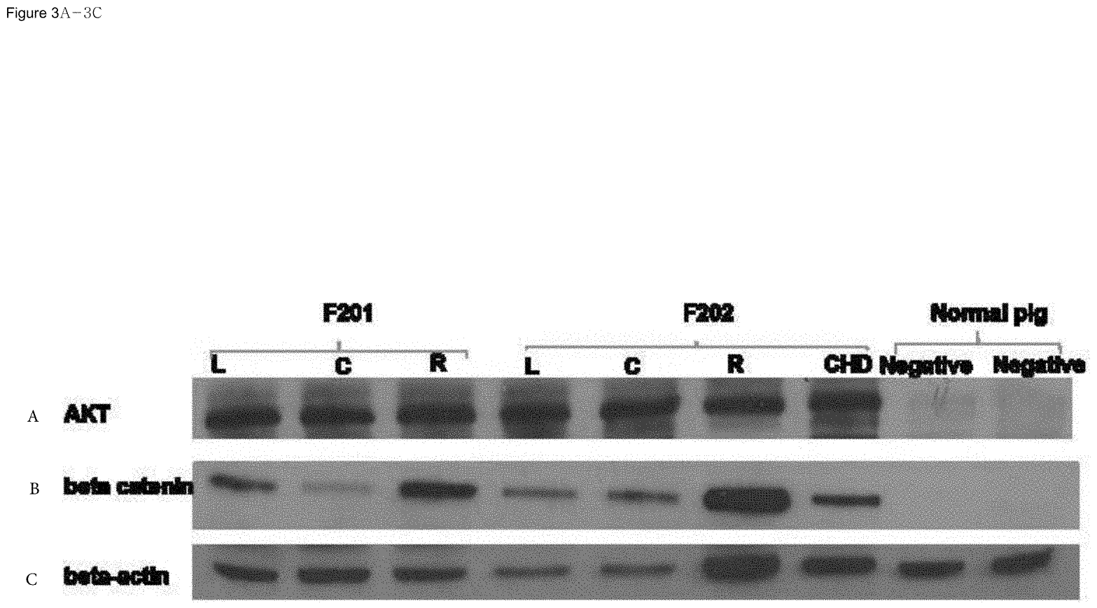

Successful and long lasting in vivo gene therapy requires DNA integration vs. episomal expression of the delivered gene. Although the inventos demonstrated successful transduction of swine liver via PCR, the inventors sought to determine whether the delivered plasmid genes were integrated and successfully replicated, transcribed and translated into functional proteins. The same four swine that underwent ERCP and hydrodynamic injection with the combination of two plasmids (AKT+beta-catenin) were used to ascertain if protein expression occurs at 60 days post procedure. The livers from these four swine were harvested and lysed, swine liver lysates were randomly collected (left, caudate, right lobes and CHD samples) and analyzed for the expression of the tagged AKT or beta-catenin by Western blot. The housekeeping gene beta-actin was utilized as internal control by Western blot. The inventors found that each analyzed liver tissue expressed the delivered AKT and beta-Catenin proteins (FIG. 3), indicating successful genomic (as opposed to episomal) integration, transcription and protein synthesis.

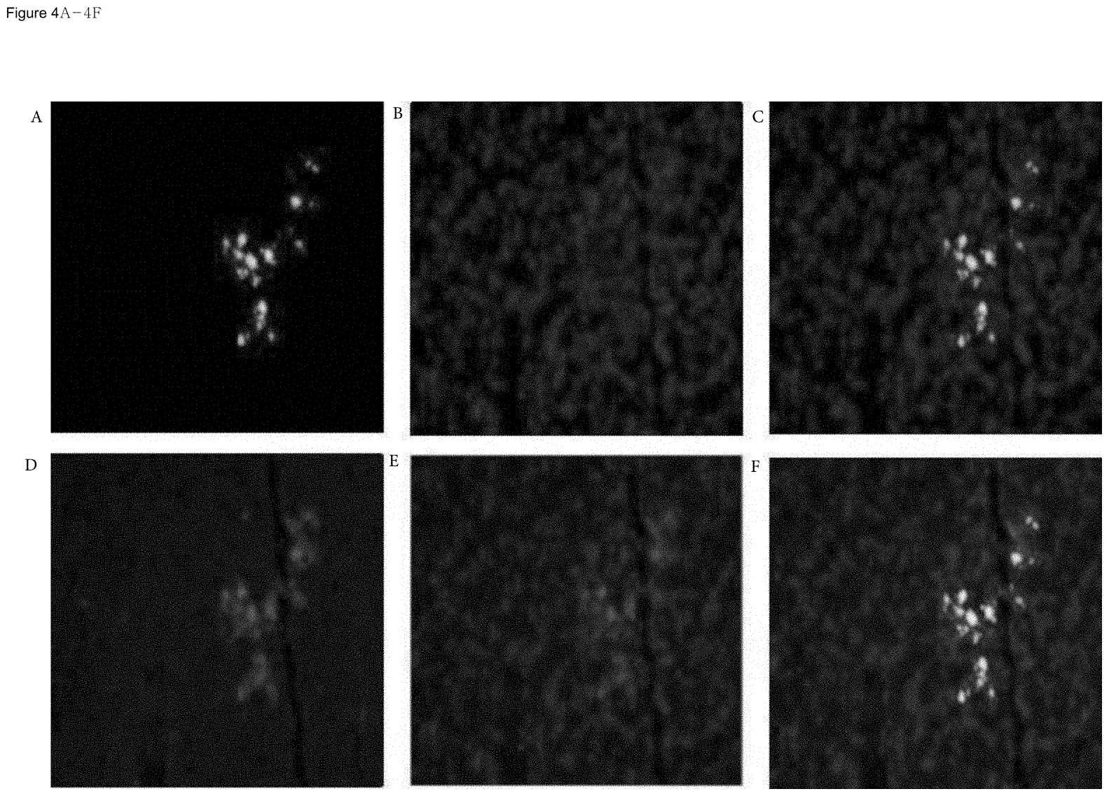

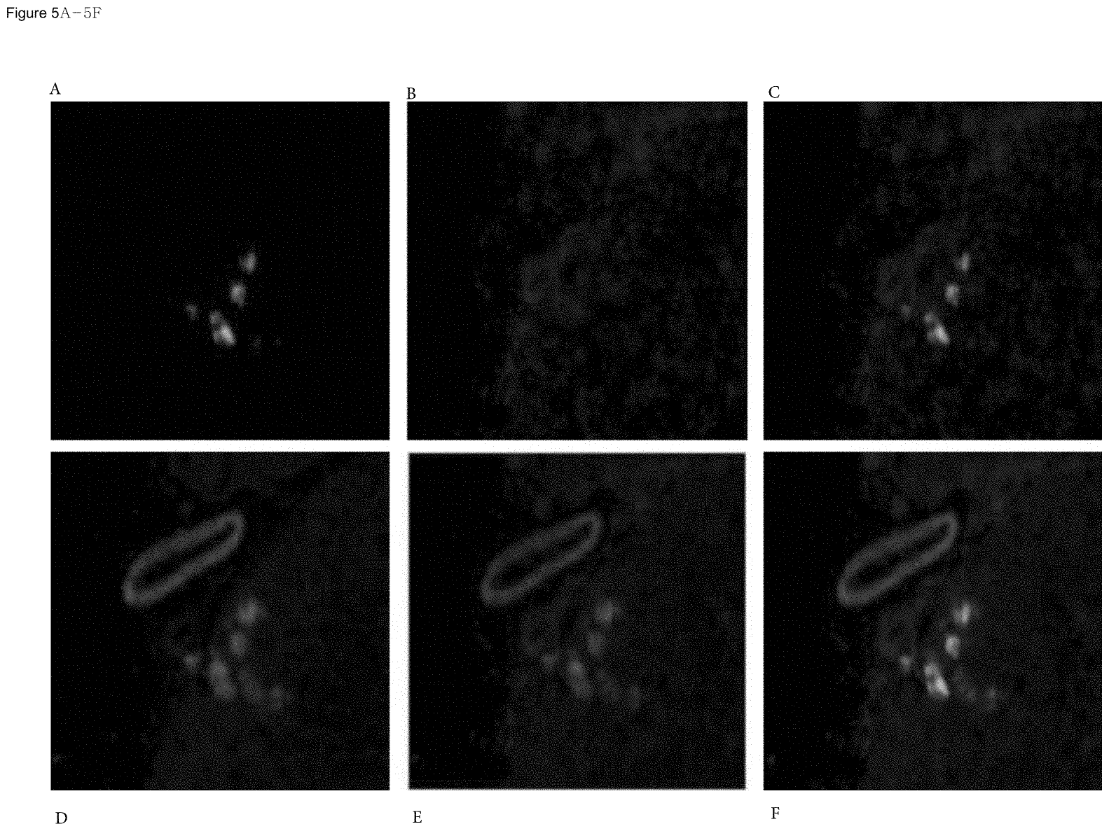

To determine the extent and exact location of the integrated gene protein expression and whether both constructs can be integrated into the same cells, liver specimen of four swine in the third group were harvested, embedded in paraffin and sectioned. These slides were stained with anti-HA-tag-AKT (green fluorescence) and MYC-tag-beta-catenin (red fluorescence). Pockets of liver tissue express both AKT and beta-catenin in the same cells (FIG. 4). In addition, beta-catenin is highly expressed in the hepatic parenchyma and nearby bile duct, while AKT only expressed in hepatic parenchyma (FIG. 5). Thus, immunostaining further confirmed that the inventors can successfully deliver target genes to the liver via ERCP hydrodynamic injection, and that the genes can integrate, replicate together and remain expressed in the liver cells long term.

To date, it has been impossible to perform liver specific hydrodynamic gene delivery in a large animal model with direct translatability to human trials. Non-rodent studies thus far have exclusively reported hydrodynamic gene delivery through the vascular system (portal, hepatic veins or IVC). Studies utilizing intravascular hydrodynamic gene delivery have demonstrated the technique to be invasive, technically challenging, cumbersome, and associated with severe cardiorespiratory compromise.¹⁶ To avoid the adverse cardiorespiratory events, “lobe specific” gene delivery has been reported with the notion that sequential targeting of several liver lobes will be necessary.¹²⁻¹⁵ To circumvent these problems, here the inventors investigated the feasibility and safety of intra-biliary hydrodynamic gene delivery.

Using an ERCP technique, the inventors could identify safe injection parameters and successfully transduce hepatocytes with genes and express the protein in liver cells. Furthermore, only relatively small volumes of plasmids were necessary to target all liver segments and no biliary or liver parenchymal injury was observed. Our results indicate that intra-biliary hydrodynamic gene delivery is minimally invasive, technically simple, and safe. Cumulatively, the data presented establishes the utility of ERCP-mediated hydrodynamically delivered liver-targeted gene therapy and indicates this technique for utilization in human patients.

We also recognized that swine (40-50 kg) have similar hepatobiliary anatomy and duct caliber to humans. In addition, similar to the anatomy of humans, the swine liver allows for two potential mechanisms to promote gene uptake by hepatocytes through intra-biliary injection. Hepatocytes can be exposed to bile duct retrograde flow through biliary canaliculi, though canalicular tight junctions might be expected to restrict DNA uptake by hepatocytes to the canalicular membrane of the hepatocyte. However, canaliculi are of sufficient size (1 gm in diameter) to permit access of plasmids to the space of Disse and subsequently be taken by the apical membrane of hepatocytes. Finally, rodent studies describing intra-biliary injection of plasmids have yielded promising results.²⁴⁻²⁶ Zhang et al.²⁵ demonstrated that plasmids had similar levels of access to the liver by the intra-biliary or portal vein route; however, plasmid complexes persisted in the liver much longer after intra-biliary delivery. Otsuka et al.³⁶ compared intra-biliary vs. portal vein liver directed gene transfer in rats and pigs using liposomes and found that transgene expression in the intra-biliary group lasted longer and lower amounts of DNA were required to achieve the same outcome.

Intra-biliary hydrodynamic gene delivery was minimally invasive, technically simple and well tolerated. Only one proceduralist and one assistant were used to perform ERCP and hydrodynamic injection. Post procedure pain was not observed without any analgesics utilized throughout the study. Additionally, no evidence of cholangitis or liver abscess was noted despite the absence of peri-procedural antibiotics. Liver biochemistry did not reveal evidence of hepatocellular or biliary epithelial injury, which is in contrast to intravascular hydrodynamic injection.¹²⁻¹⁵ Although at this point the inventors can only hypothesize that this procedure will be equally benign in human patients, it appears that its safety profile may not be a barrier to clinical trials. It must be acknowledged that in swine models, scenarios such as failed biliary cannulation and post-ERCP pancreatitis are extremely unlikely as the pancreatic orifice is several centimeters downstream from the biliary orifice. Although there are potential adverse events associated with ERCP in the clinical setting (including the pediatric population), it remains a widely utilized procedure.³⁷⁻³⁹ Furthermore, if there is suboptimal efficiency of transduction, one can consider performing repeated hydrodynamic injections over the course of treatment. The procedure time and the risk of ERCP related adverse events are significantly reduced in the subsequent procedure. Endoscopic ultrasound guided biliary access is disseminating and likely reduces the risk of post ERCP pancreatitis. However, it would be challenging to create a closed system and generate the hydrodynamic pressures necessary with current needle systems.

Despite successful and safe transduction of plasmids throughout the liver, there is room for further investigation into our technique. In the current study, the inventors did not measure the pressure generated within the biliary tree and our methods were geared to identifying the maximal tolerable pressure. Identifying the minimal pressure parameters for transduction will increase the safety. Second, although the inventors demonstrated 100% plasmid DNA transduction, the inventors did not assess the efficiency of transduction as a function of procedural parameters, such as pressure, volume, and time of delivery or plasmid concentration. Third, the inventors did not assess for non-target (heart etc.) delivery of plasmids, although conceivably less of a problem compared to the vascular route. However, even if plasmid DNA escaped the liver and circulated to a different organ, there would be no pressure applied in the distant organ that would force the plasmids past the cell membrane. Therefore, it is tempting to hypothesize that this technique would have no clinically relevant distant gene transduction. Fourth, this technique may need to be adapted to patients who already are significantly diseased (i.e. liver cirrhosis). Fifth, the inherent limitations of studies performed in animals mandate careful consideration prior to validation in human patients.

Hydrodynamic gene delivery via the bile duct represents an important step towards the clinical use of non-viral gene delivery. Herein the inventors demonstrate that a minimally invasive, low cost and safe technique was able to transduce hepatocytes throughout the liver with a single injection. Furthermore, the inventors demonstrate that effective DNA integration and protein production in the liver of large animals of hydrodynamically delivered plasmids is feasible without systemic side effects. The clinical applications made possible by this approach will depend on the precise localization of gene expression (hepatocytes, cholangiocytes, sinusoidal epithelial cells), the efficiency of gene transfer (proportion of cells expressing the gene) and the longevity of gene expression. Clarification of the aforementioned outcomes will be necessary prior to clinical utilization of intra-biliary hydrodynamic gene delivery.

### Example 2: Human Factor IX Gene Therapy

Endoscopic equipment currently used in human patients was employed. Using similar parameters to our previous study, ERCP-guided hydrodynamic injection was performed in four Yorkshire pigs (˜40 kg). After confirming catheter placement with contrast, a balloon was inflated in the common hepatic duct to seal the system. 30 mL of DNA solution (3-5.5 mg plasmid DNA in 0.9% NaCl) was injected over 15 seconds by power injector providing a significant increase in volume and pressure within the intrahepatic biliary system, which normally holds ˜5 mL of bile.

### Results

The procedure was completed within 43±11 minutes across 4 pigs. During the hydrodynamic injection, no significant change in vital signs (blood pressure, heart rate, respiration rate, SpO₂ and end-tidal CO₂) was noted. All four pigs exhibited normal behavior post-procedure with no acute clinical signs or distress in the hours. Post-procedure bedside ultrasound showed no hematoma nor dilation of the common hepatic duct or common bile duct. Similarly, fluoroscopy showed intact biliary tree with no dilation or rupture from fluid overload or pressure. CBC showed no acute inflammatory response in the days post-procedure, and no decrease in hemoglobin reflecting bleeding was noted. Post-injection liver enzyme panel to test for hepatocyte damage showed mildly elevated ALT at 49±9 U/L on day 1 and 50±11 U/L on day 4 (normal range: 22-47 U/L), respectively, while AST and albumin were within normal range at those time points. Moreover, GGT remained within normal range indicating no cholestasis. Total and direct bilirubin were within normal range at time points post-procedure, indicating a lack of bile leakage from the biliary system to vascular compartment.

Conclusion

Pigs tolerated endoscopic hydrodynamic injection into their intrahepatic biliary system well with no acute hemodynamic side effects, bile duct injury or significant hepatocyte damage observed. Given that pigs have similarly sized organs and anatomy to human patients, the data indicates that hydrodynamic gene delivery into liver could be similarly safe when used clinically for human patients.

### Methods

A transgene cassette featuring a liver-specific promoter based on alpha-antitrypsin, along with an intron to enhance expression. Hyperactive piggyBac transposase was used to integrate in hFIX expression cassette, affording stable production and avoiding plasmid silencing.

### Procedure

Four female pigs ˜40 kg were injected.
Pig 1: The first injection only had 26 mL due to greater than expected deadspace being observed. 3 mg FIX transposon, 0.4 mg hyperPB transposase
Pig 2: The second pig received a full 30 mL. 3 mg FIX transposon, 0.4 mg hyperPB transposase
Pig 3: The third pig received a full 30 mL. 5.5 mg FIX transposon, 0.6 mg hyperPB transposase
Pig 4: The fourth pig received a 25-26 mL. 5.5 mg FIX transposon, 0.6 mg hyperPB transposase

### Summary of Data

Blood samples were collected: pre-procedure, 10 minutes post-procedure, Day 1 post procedure, Day 4 post procedure. The following was observed:
No acute toxicities from the injection of ˜30 ml over 2 mL/sec at 999 psi in the common hepatic duct of the biliary tree; time points where pre-procedure and post-procedure (10 minutes);
ALT/AST within normal range;
No change in direct or total bilirubin;
GGT, albumin, LDH also largely normal;
Subsequent labs at Day 1 and Day 4 were similarly normal;
No acute abnormalities from the injection as defined by ultrasound during the injection on the procedure day; size of the gall bladder stayed the same.

In these further pig trials, steady-state intrabiliary pressure during injection ranged between 50-150 mmHg depending on injection parameters. Pressures provided by the inflated extraction balloon ranged between 10-20 mmHg. See conditions utilized set forth in Table 2 below.

TABLE 2

| | | | | Steady-State | Pressure Before |
|---|---|---|---|---|---|
| | Volume | Rate of | Pressure After | Pressure During | Deflating |
| | Administered | Injection | Initial Injection | Injection | Balloon |
| Trial Name | (mL) | (mL/sec) | (mmHg) | (mmHg) | (mmHg) |
| Pig #2, Trial 1 | 50 | 3 | 181.36 | 148.58 | 18.92 |
| Pig #3, Trial 1 | 30 | 2 | 46.08 | 36.42 | 10.71 |
| Pig #3, Trial 2 | 30 | 2 | 89.12 | 85.06 | 4.23 |
| Pig #3, Trial 1 | 140 | 1 | 114.76 | 82.49 | 9.98 |

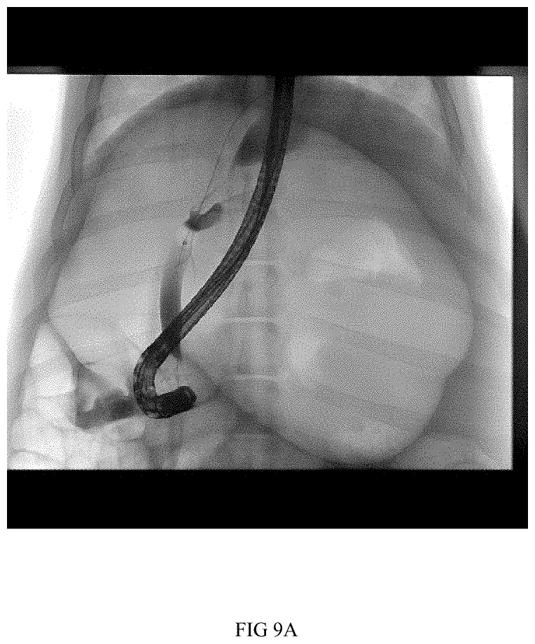

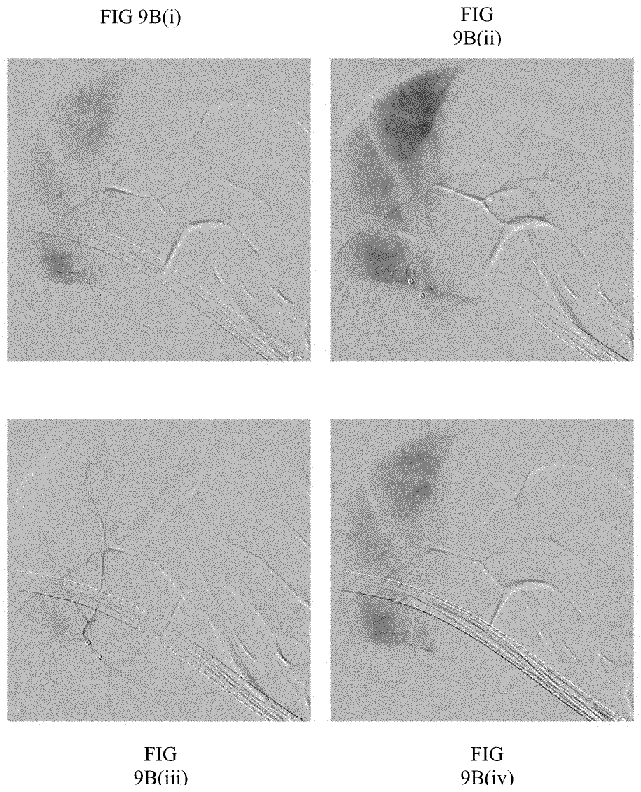

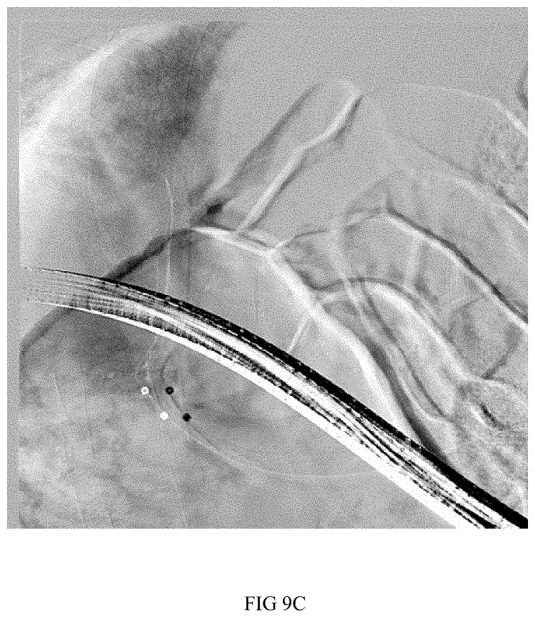

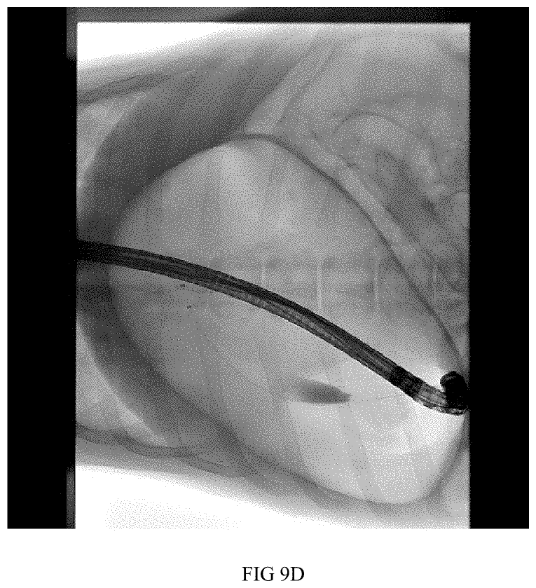

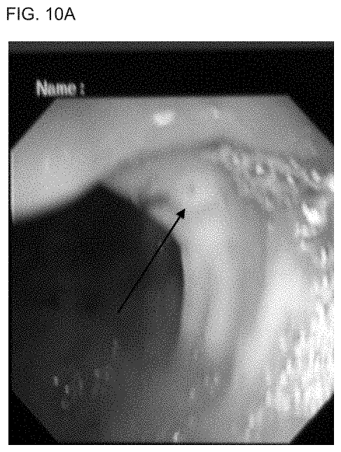

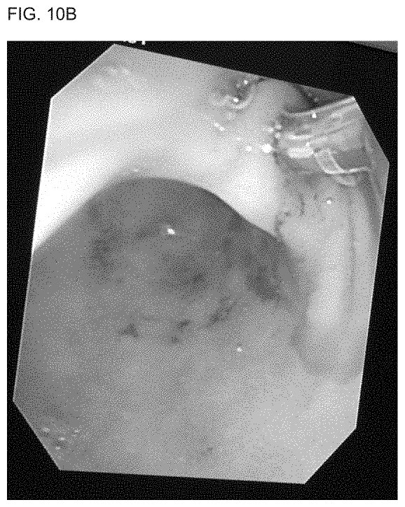

In FIG. 9, procedures for the above protocols are shown. Thus in FIG. 9A: Normal biliary anatomy. The duodenoscope is used to access the papilla through a retrobiliary route. Contrast is injected and the biliary tree opacifies. The cystic duct and gallbladder opacify as does the common bile duct and the common hepatic duct. The balloon is inflated but is below the level of the cystic duct so the hydrodynamic injection is NOT performed with the balloon catheter in this location. The balloon catheter must be above the cystic duct so that material is not inadvertently injected into the gallbladder.

FIG. 9B (9B(i) though 9B(iv)): Images from left to right and top to bottom. This experiment shows what our method of hydrodynamic injection does when contrast is used (as opposed to genetic material). Although contrast likely did also “escape” into the venous system draining the liver, alarge portion did enter the hepatocytes. FIG. 9B(i): Balloon catheter with balloon inflated and contrast injected under pressure (1 sec of injection). FIG. 9B(ii): Acinarization of the left and right lobes of the liver after 2-4 seconds. FIG. 9B(iii): Acinarization of the left and right lobes of the liver after 4-6 seconds. FIG. 9B(iv): Acinarization of the left and right lobes of the liver after 8-10 seconds.

FIG. 9C: Another image of significant acinarization throughout the liver. Although it looks like 2 catheters are present, there is only one and this is respiratory artifact

FIG. 9D: Balloon catheter in the common hepatic duct (above the cystic duct) and hence in the optimal position for hydrodynamic injection.

### Example 3: Further Nucleic Acid Delivery

1. Case 1: Pig #503

(A) A first injection of 10 cc was injected probably in the cystic duct of the pig subject, the working channel of the balloon catheter was primed with 1.5 cc of contrast. A 10 cc plasmids was connected to the injection port of the cook fusion balloon catheter (8.5-15 mm, short wire so the guidewire port was nit used and the injection port was used) without inflating the balloon. 10 cc of plasmids was injected and it appeared the catheter was situated in the cystic duct as opposed to the right hepatic duct.
(B) The left hepatic duct was cannulated & the balloon was inflated to 12 mm and 10 cc of plasmids was injected into the left hepatic duct over 5 seconds,
(C) The middle hepatic duct was then cannulated and the balloon was inflated to 12 mm and 10 cc of plasmids was injected over 5 seconds.

On each occasion the balloon was kept inflated for 2 minutes.

In this pig the pancreatic duct could not be identified despite intensive interrogation with the duodenoscope and colonoscope.

2. Case 2: Pig #501.

(A) Balloon catheter was placed into the right hepatic duct of the pig subject, was initially inflated to 15 mm but then decreased to 12 mm as the large balloon could be seen on fluoroscopy. Then 10 cc of plasmids was injected over 5 seconds.
(B) The left hepatic duct was cannulated. the balloon was inflated to 12 mm and 10 cc of plasmids was injected over 5 seconds.
(C) The pancreatic duct was identified and balloon was situated near the orifice within the pancreatic duct and was inflated to 12 mm followed by 10 cc of plasmids injected over 5 seconds.

On each occasion the balloon was kept inflated for 2 minutes.

3. Case 3: Pig #502

(A) A balloon catheter was placed in the Right hepatic duct of the pig subject, and the balloon was inflated to 12 mm, well visualized through fluoroscopy followed by a 10 cc injection of plasmids, injected over 5 seconds.
There was an initial debate as to weather the catheter was in the cystic duct or right hepatic, but as the guide wire did not coil and was able to be deeply inserted to the edge of the liver we felt it was highly unlikely in the cystic duct.
(B) The Left hepatic duct was inflated with a balloon catheter, inflated to 12 mm and well visualized. A 10 cc plasmids injection was injected over 5 seconds.
(C) The pancreatic duct was well cannulated and 12 mm balloon catheter was inflated, followed by a 10 cc of plasma injected over 5 seconds.

On each occasion the balloon was kept inflated for 2 minutes.

For each of the above Cases 1-3 the plasmid used was c-myc+NRAS+AKT 25 ug/ml in saline, and add SB at the ratio of 1:25 oncogene.

4. Case 4: Pig #520

(A) A balloon catheter was placed in the right hepatic duct of the pig subject, and was inflated to 12 mm, well visualized through fluoroscopy, followed by a 10 cc injection of plasmids pushed in over 5 seconds.
(B) next the middle hepatic duct was identified and balloon catheter was inflated, followed by infusion of 10 cc of plasma, in 5 seconds.
(C) After extensive exploration the pancreatic duct was identified and cannulated. a 12 mm balloon was inflated followed by infusion of plasmid of 10 cc of plasma over 5 seconds.

On each occasion the balloon was kept inflated for 2 minutes.

The endosocope was chewed by the pig as it awoke inadvertently. The scope shaft is extensively damaged. The endoscope remains functional though I will arrmage for it to be sent for repair.

5. Case 5: Pig #521

The balloon catheter was placed in the left middle hepatic duct of the pig subject, and inflated to 12 mm, followed by infusion of 10 cc plasmids over 5 seconds. The balloon was kept inflated for 2 minutes.

In this pig the pancreatic duct and right hepatic duct were not identified inspite of extensive exploration. The guidewire and catheter at one point were in the right system but the unstable endoscope position in this larger pig meant that the position was lost.

6. Case 6: Pig #519

(A) The balloon catheter was placed in the left hepatic duct of the pig subject, and inflated to 12 mm, followed by infusion of 10 cc plasmids over 5 seconds.
(B) The middle hepatic lobe was identified and 12 m balloon was inflated, followed by a 10 cc infusion of plasmids over 5 seconds.
(C) The pancreatic duct was visualized and cannulated, a 12 mm balloon was inflated, followed by 10 cc of plasmids infusion in 5 seconds.

On each occasion the balloon was kept inflated for 2 minutes.

For each of the above Cases 4-6 the plasmid used was NRNAS+AKT 25 ug/ml in saline, and add SB at the ration of 1:25 oncogene.

7. Case 7# Pig E 627.

A balloon catheter was placed in the left hepatic duct of the pig subject, and was inflated to 12 mm, followed by infusion of 10 cc plasmids over 5 seconds. The balloon was kept inflated for 2 minutes.

3 cc of contrast was injected in what could have been the right hepatic duct or cystic duct, since we were unsure we did not inject the plasmids. Therefore we did not inject plasmids into the right system as if we were in the cystic duct we may rupture it as we inflate the balloon.

In spite of great endoscopic visualization we were unable to find the pancreatic orifice.

8. Case 8# Pig E 625

(A) A balloon catheter was placed in the right middle hepatic duct of the pig subject, and was inflated to 12 mm, well visualized through fluoroscopy, followed by a 10 cc injection of plasmids pushed in over 5 seconds.
(B) Next the left hepatic duct was identified and balloon catheter was inflated, followed by infusion of 10 cc of plasma, in 5 seconds.
(C) After extensive exploration the pancreatic duct was identified and cannulated. a 12 mm balloon was inflated with the catheter just inside the pancreatic orifice followed by infusion of plasmid of 10 cc of plasma over 5 seconds.
On each occasion the balloon was kept inflated for 2 minutes.

### 9. Case 9 # Pig E 626

(A) A balloon catheter was placed in the right middle hepatic duct of the pig subject, and was inflated to 12 mm, well visualized through fluoroscopy, followed by a 10 cc injection of plasmids pushed in over 5 seconds.
(B) Next the left hepatic duct was identified and balloon catheter was inflated, followed by infusion of 10 cc of plasmids, in 5 seconds.
(C) After extensive exploration the pancreatic duct was identified and cannulated. the wire in the duct appeared to take an unusual route, so to confirm that we were in the PD we injected approximately 3 ml contrast to make sure we were intraductal and once confirmed, a 12 mm balloon was inflated followed by infusion of 10 cc of plasmids over 5 seconds.

In this pig we also identified the CBD and without the balloon inflation, we injected 10 cc of plasmids over 5 seconds. This was done solely as a trial as we had 10 cc of plasmids remaining.

On each occasion, where required the balloon was kept inflated for 2 minutes.

For each of the above Cases 7-9 the plasmid used was Type of plasmid used: (PT3EFla-AKT)250 ug+ PT3EF1a-YAP 250 ug+pCMV-SB 20 ug saline in 10 ml saline.

### Example 4: Further Nucleic Acid Delivery—Pancreatic Duct

A nucleic acid composition was prepared with cMyc 600 ug+SB 24 ug to make 60 ml of solution.
Conc=10.4 ug/ml. Then nanoparticles added.

### Pancreatic Duct Hydro:

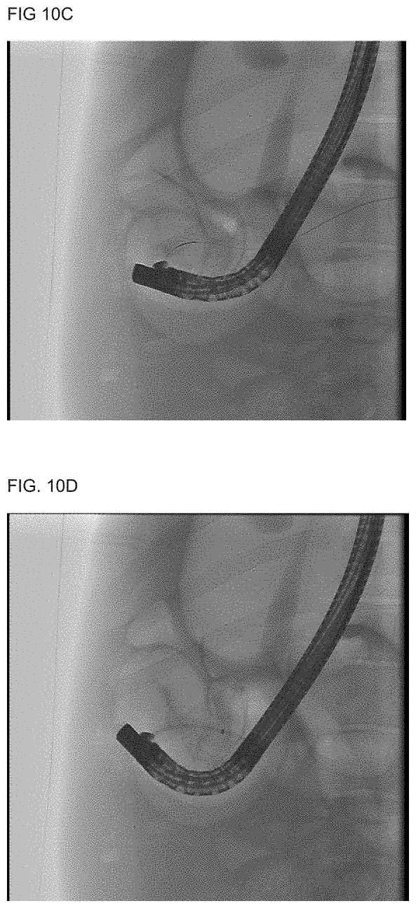

100 lbs pig was used as a subject. The pancreatic duct was identified 10-15 cm distal to the bile duct after the 1^{st} corner on the upper right hand side (with a duodenoscope). This and cannulated with a sphincterotome and guidewire and then exchanged for a balloon catheter. The Balloon was inflated to 9 mm and 7 ml plasmid was injected under pressure after the catheter had been primed. No resistance noted. Balloon was deflated after 1 min and catheter was withdrawn after 1 min. No bleeding was observed at the end of the procedure. Aspects of the procedure are shown in FIGS. 10A-10D.

### REFERENCES

1. Flotte T R, Trapnell B C, Humphries M, et al. Phase 2 clinical trial of a recombinant adeno-associated viral vector expressing alphal-antitrypsin: interim results. Hum Gene Ther 2011; 22:1239-47.
2. Bogliolo M, Surralles J. Fanconi anemia: a model disease for studies on human genetics and advanced therapeutics. Curr Opin Genet Dev 2015; 33:32-40.
3. Lehrman S. Virus treatment questioned after gene therapy death. Nature 1999; 401:517-8. Raper S E, Yudkoff M, Chirmule N, et al. A pilot study of in vivo liver-directed gene transfer with an adenoviral vector in partial ornithine transcarbamylase deficiency. Hum Gene Ther 2002; 13:163-75.
5. Aronovich E L, Mclvor R S, Hackett P B. The Sleeping Beauty transposon system: a non-viral vector for gene therapy. Hum Mol Genet 2011; 20:R14-20.
6. Garels W, Mates L, Holler S, et al. Germline transgenic pigs by Sleeping Beauty transposition in porcine zygotes and targeted integration in the pig genome. PLoS One 2011; 6:e23573.
7. Maiti S N, Huls H, Singh H, et al. Sleeping beauty system to redirect T-cell specificity for human applications. J Immunother 2013; 36:112-23.
8. Yant S R, Meuse L, Chiu W, et al. Somatic integration and long-term transgene expression in normal and haemophilic mice using a DNA transposon system. Nat Genet 2000; 25:35-41.
9. Ohlfest J R, Frandsen J L, Fritz S, et al. Phenotypic correction and long-term expression of factor VIII in hemophilic mice by immunotolerization and nonviral gene transfer using the Sleeping Beauty transposon system. Blood 2005; 105:2691-8.
10. Mikkelsen J G, Yant S R, Meuse L, et al. Helper-Independent Sleeping Beauty transposon-transposase vectors for efficient nonviral gene delivery and persistent gene expression in vivo. Mol Ther 2003; 8:654-65.
11. Aravalli R N, Belcher J D, Steer C J. Liver-targeted gene therapy: Approaches and challenges. Liver Transpl 2015; 21:718-37.
12. Kamimura K, Kanefuji T, Yokoo T, et al. Safety assessment of liver-targeted hydrodynamic gene delivery in dogs. PLoS One 2014; 9:e107203.
13. Kamimura K, Suda T, Xu W, et al. Image-guided, lobe-specific hydrodynamic gene delivery to swine liver. Mol Ther 2009; 17:491-9.
14. Kamimura K, Suda T, Zhang G, et al. Parameters Affecting Image-guided, Hydrodynamic Gene Delivery to Swine Liver. Mol Ther Nucleic Acids 2013; 2:e128.
15. Khorsandi S E, Bachellier P, Weber J C, et al. Minimally invasive and selective hydrodynamic gene therapy of liver segments in the pig and human. Cancer Gene Ther 2008; 15:225-30.
16. Zhang G, Gao X, Song Y K, et al. Hydroporation as the mechanism of hydrodynamic delivery. Gene Ther 2004; 11:675-82.
17. Xue W, Chen S, Yin H, et al. CRISPR-mediated direct mutation of cancer genes in the mouse liver. Nature 2014; 514:380-4.
18. Vakili S, Ebrahimi S S, Sadeghi A, et al. Hydrodynamic-based delivery of PTPIB shRNA reduces plasma glucose levels in diabetic mice. Mol Med Rep 2013; 7:211-6.
19. Hibbitt O C, Harbottle R P, Waddington S N, et al. Delivery and long-term expression of a 135 kb LDLR genomic DNA locus in vivo by hydrodynamic tail vein injection. J Gene Med 2007; 9:488-97.
20. Feng D M, He C X, Miao C Y, et al. Conditions affecting hydrodynamics-based gene delivery into mouse liver in vivo. Clin Exp Pharmacol Physiol 2004; 31:850-5.
21. Hagstrom J E. Plasmid-based gene delivery to target tissues in vivo: the intravascular approach. Curr Opin Mol Ther 2003; 5:338-44.
22. Zhang G, Vargo D, Budker V, et al. Expression of naked plasmid DNA injected into the afferent and efferent vessels of rodent and dog livers. Hum Gene Ther 1997; 8:1763-72.
23. Racz Z, Godo M, Revesz C, et al. Immune activation and target organ damage are consequences of hydrodynamic treatment but not delivery of naked siRNAs in mice. Nucleic Acid Ther 2011; 21:215-24.
24. Hu J, Zhang X, Dong X, et al. A remarkable permeability of canalicular tight junctions might facilitate retrograde, non-viral gene delivery to the liver via the bile duct. Gut 2005; 54:1473-9.
25. Zhang X, Collins L, Sawyer G J, et al. In vivo gene delivery via portal vein and bile duct to individual lobes of the rat liver using a polylysine-based nonviral DNA vector in combination with chloroquine. Hum Gene Ther 2001; 12:2179-90.
26. Chen C Y, Liu H S, Lin X Z. Hydrodynamics-based gene delivery to the liver by bile duct injection of plasmid DNA—the impact of lasting biliary obstruction and injection volume. Hepatogastroenterology 2005; 52:25-8.
27. Ajiki T, Onoyama H, Yamamoto M, et al. Detection of point mutations in K-ras gene at codon 12 in bile from percutaneous transhepatic choledochal drainage tubes for diagnosis of biliary strictures. Int J Pancreatol 1995; 18:215-20.
28. Stauffer J K, Scarzello A J, Andersen J B, et al. Coactivation of AKT and beta-catenin in mice rapidly induces formation of lipogenic liver tumors. Cancer Res 2011; 71:2718-27.
29. Fan B, Malato Y, Calvisi D F, et al. Cholangiocarcinomas can originate from hepatocytes in mice. J Clin Invest 2012; 122:2911-5.
30. Calvisi D F, Wang C, Ho C, et al. Increased lipogenesis, induced by AKT-mTORC1-RPS6 signaling, promotes development of human hepatocellular carcinoma. Gastroenterology 2011; 140:1071-83.
31. Animal Welfare Act as Amended Volume 7 USC § 2131-2159, 2008.
32. Animal Welfare Regulations. Volume 9 CFR § 1-4, 2013.
33. Institute for Laboratory Animal Research. Guide for the Care and Use of Laboratory Animals 8th Edition: National Academies Press, 2011.
34. Swindle M M. Swine in the Laboratory: Surgery, Anesthesia, Imaging and Experimental Techniques, 2nd Ed. Boca Raton (Fla.): Taylor and Francis Group, 2007.
35. Liu F, Song Y, Liu D. Hydrodynamics-based transfection in animals by systemic administration of plasmid DNA. Gene Ther 1999; 6:1258-66.
36. Otsuka M, Baru M, Deriviere L, et al. In vivo liver-directed gene transfer in rats and pigs with large anionic multilamellar liposomes: routes of administration and effects of surgical manipulations on transfection efficiency. J Drug Target 2000; 8:267-79.
37. Yachimski P S, Ross A. The Future of Endoscopic Retrograde Cholangiopancreatography. Gastroenterology 2017; 153:338-344.
38. Luo H, Zhao L, Leung J, et al. Routine pre-procedural rectal indometacin versus selective post-procedural rectal indometacin to prevent pancreatitis in patients undergoing endoscopic retrograde cholangiopancreatography: a multicentre, single-blinded, randomised controlled trial. Lancet 2016; 387:2293-2301.
39. Rosen J D, Lane R S, Martinez J M, et al. Success and safety of endoscopic retrograde cholangiopancreatography in children. J Pediatr Surg 2017; 52:1148-1151.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of transfecting cells of a subject in vivo, comprising:
administering at elevated pressure an effective amount of a nucleic acid expression cassette to the subject's biliary tree, liver, kidney or pancreas.

2. The method of claim 1 wherein the elevated pressure is utilized to induce transfection in vivo.

3. The method of claim 1 or 2 wherein the elevated pressure is measured during administering of the nucleic acid expression cassette.

4. The method of any one of claims 1 through 3 wherein the elevated pressure is measured prior to and/or modified during and/or subsequent to the administering based on real-time measuring of the elevated pressure.

5. The method of any one of claims 1 through 4 wherein the nucleic acid expression cassette is delivered into a substantially closed system.

6. The method of any one of claims 1 through 4 wherein the nucleic acid expression cassette is delivered into a substantially closed system to include measuring of end results and/or modifying or injection parameters.

7. The method of any one of claims 1 through 6 wherein the administering conditions are selected based on one or more of 1) measured volume of subject's biliary tree; and 2) measured target organ characteristics.

8. The method of any one of claims 1 through 7 wherein the nucleic acid expression cassette is administered to liver, pancreas and/or kidney of a subject.

9. The method of any one of claims 1 through 8 wherein the nucleic acid expression vector is administered to hepatic or pancreatic ducts.

10. A method of transfecting cells of a subject in vivo, comprising:
administering at elevated pressure an effective amount of a nucleic acid expression cassette to the subject,
wherein the elevated pressure is measured in real-time during and/or subsequent to administering of the nucleic acid expression cassette.

11. The method of claim 10 wherein the elevated pressure is modified during and/or subsequent to the administering based on real-time measuring of the elevated pressure.

12. The method of claim 10 or 11 wherein the nucleic acid expression cassette is delivered into a substantially closed system.

13. The method of any one of claims 10 through 12 wherein the administering conditions are selected based on one or more of 1) measured volume of subject's biliary tree; and 2) measured target organ characteristics.

14. The method of any one of claims 10 through 13 wherein the nucleic acid expression cassette is administered to hepatic or pancreatic ducts.

15. A method of treating or preventing liver or pancreas disease by genetic therapy comprising the steps of:
delivering a vector comprising a nucleic acid sequence that ameliorates a liver or pancreas disease to a subject having or prone of getting a liver or pancreas disease,
creating a substantially closed space;
injecting the vector under required pressure and quantity into the closed space so that the vector is transferred to the cytoplasm of cells of the liver or pancreas;
expressing the nucleic acid sequence and treating or preventing a liver or pancreas disease in the subject.

16. The method of claim 15 further comprising:
providing an endoscopic retrograde cholangiopancreatography (ERCP) comprising the vector and a balloon;
positioning the endoscope through the mouth, the stomach, duodenum, and the duct of the pancreas or the liver of the subject;
opening the balloon to thereby create the substantially closed space.

17. The method of claim 15 or 16 wherein the nucleic acid sequence encodes a protein or a peptide.

18. The method of any one of claim 15 through 17 wherein the nucleic acid encodes a second nucleic acid selected from the group consisting of an shRNA, mRNA and a combination thereof.

19. The method of any one of claims 15 through 18 wherein the vector is selected from the group of a virus or a plasmid.

20. The method of claim 19 wherein the virus is AAV.

21. The method of any one of claims 15 through 20 wherein the nucleic acid sequence encodes a protein that treats cirrhosis or a functional part thereof.

22. The method of any one of claims 15 through 21 wherein the nucleic acid sequence encodes a protein that treats a pancreas disease or a functional part thereof.

23. The method of any one of claims 15 through 22 wherein the plasmid comprises an SB transposon comprising the nucleic acid sequence.

24. The method of any one of claims 15 through 23 further comprising the step of removing the remaining vector through the endoscope.

25. The method of any one of claims 15 through 24 wherein the protein or peptide is an antibody.
